(19) United States
(12) Patent Application Publication
(10) Pub. No.: US 2026/0097152 A1
(43) Pub. Date: Apr. 9, 2026

(54) MEDICAL DEVICE WITH MULTI-LAYER COATING INCORPORATING CATIONIC STEROIDAL ANTIMICROBIALS

(71) Applicant: Paul B. Savage, Mapleton, ID (US)

(72) Inventor: Paul B. Savage, Mapleton, ID (US)

(73) Assignee: BRIGHAM YOUNG UNIVERSITY, Provo, UT (US)

(21) Appl. No.: 18/906,723

(22) Filed: Oct. 4, 2024

(51) Int. Cl. A61L 29/16

(52) U.S. Cl. CPC A61L 29/16; A61L 2300/404

(57) ABSTRACT

Disclosed is a multi-layered coating for application to an implantable medical device. The multi-layered coating comprises (i) a CSA coating layer comprising a polymer in which one or more CSA compounds are distributed, and (ii) a top coating layer disposed over the CSA coating layer, the top coating layer being free of CSA compounds. In use, the multi-layered coating is applied to a base substrate forming at least a portion of the implantable medical device. When the implantable medical device is exposed to a physiological environment, the top coating layer provides (i) more sustained elution of the one or more CSA compounds, as compared to the implantable medical device without the top coating layer, and/or (ii) reduced swelling of the CSA coating layer, as compared to the implantable medical device without the top coating layer.

### BACKGROUND

An ongoing problem for hospitals and other healthcare facilities is the threat of nosocomial or healthcare acquired infections (HAIs). According to the World Health Organization's (WHO) report on infection prevention and control, 7-15 hospitalized patients in 100 will acquire at least one HAI, with 1 in every 10 infected patients dying from the infection. Notably, during and since the COVID-19 pandemic, HAIs have been on the rise, and due to increasing antibiotic resistance, infections are becoming more dangerous.

Antibiotic resistance is a worsening global crisis arising from use and misuse of antibiotics and lack of newer drugs, and as many as 46% of bacteria from healthcare or other hygienic facilities are multi-drug resistant (MDR). Compounding this problem is the prevalence of medical device-related infections. For example, despite the use of antibiotics, as many as 2.8% of patients using a peripherally inserted central catheter (PICC) line suffer bloodstream infections.

Accordingly, there is an ongoing need for antimicrobial approaches that can reduce the incidence and/or severity of medical device-related infections.

### SUMMARY

Disclosed herein is a multi-layered coating for application to an implantable medical device and implantable medical devices with such coating. The multi-layered coating comprises (i) a cationic steroidal antimicrobial (CSA) coating layer comprising a polymer in which one or more CSA compounds are distributed, and (ii) a top coating layer disposed over the CSA coating layer, the top coating layer being free of CSA compounds. In use, the multi-layered coating is applied to a base substrate forming at least a portion of the implantable medical device. When the implantable medical device is exposed to a physiological environment, the top coating layer provides (i) more sustained elution of the one or more CSA compounds, as compared to the implantable medical device without the top coating layer, and/or (ii) reduced swelling of the CSA coating layer, as compared to the implantable medical device without the top coating layer.

Also disclosed herein is an implantable medical device configured to elute a CSA compound upon exposure to a physiological environment. The implantable medical device includes a base substrate forming at least a portion of the implantable medical device, and the multi-layered coating disposed on the base substrate.

Also disclosed herein is a method of manufacturing an implantable medical device configured to elute one or more CSA compounds, the method comprising: applying a CSA coating layer over a base substrate, the base substrate forming at least a portion of the implantable medical device, the CSA coating layer comprising a polymer in which one or more CSA compounds are distributed; and applying a top coating layer over the CSA coating layer, the top coating layer being free of CSA compounds.

Also disclosed herein is a method of using the multi-layered coating composition to limit microbial growth (planktonic and/or biofilm) on an implantable medical device.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the disclosure will become apparent and more readily appreciated from the following description, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1A illustrates examples of cationic steroidal antimicrobial (CSA) compounds having ester or amide linkages at one or more of the R₃, R₇, and R₁₂ positions;

FIG. 1B illustrates examples of CSA compounds having ether linkages at one or more of the R₃, R₇, and R₁₂ positions;

FIG. 1C illustrate example CSA compounds having an amide linkage within the R₁₈ group and ether or urethane linkages at the R₃, R₇, and R₁₂ positions;

FIG. 1D illustrate example CSA compounds having a glyceride linkage within the R₁₈ group and ester linkages at the R₃, R₇, and R₁₂ positions.

FIGS. 2A-2F show characterization of three coating systems: Coating A samples have a 6 μm polyurethane coating containing 20% (w/w) CSA-131NDSA; Coating B samples have an 18 μm polyurethane coat containing 50% (w/w) CSA-131NDSA; Coating C samples have an 18 μm polyurethane CSA coating layer containing 50% (w/w) CSA-131NDSA with a 12 μm polyurethane top coating layer. FIG. 2A: Antimicrobial efficacy assay of Coating A against repeated inoculations with MRSA. Experiments run in triplicate. * p<05. FIG. 2B: Total extraction of CSA-131 from triplicates of Coating A and Coating B. FIG. 2C: Representative elution profiles of CSA-131 from Coating B and Coating C. FIG. 2D: SEM image taken of uncoated PICC line surface. FIG. 2E: SEM image taken of a PICC line surface coated with Coating B. Defects from handling, located along the lower edge, were included to facilitate focusing. FIG. 2F: SEM image of PICC line cross section of Coating C. Frontiers between coating layers are visible and highlighted while layers are labeled for clarity. Error bars represent standard deviation.

FIGS. 3A-3D: Efficacy of PICC line segments with Coating C against planktonic pathogens. Quantification of planktonic pathogen after daily challenges to uncoated (control) and coated PICC line segments by MRSA (FIG. 3A), P. aeruginosa (FIG. 3B), K. pneumoniae (FIG. 3C), and C. albicans (FIG. 3D). All experiments were run in triplicate. Error bars represent standard deviation. * p<0.05.

FIGS. 4A-4F: Efficacy of coated PICC line segments against biofilm formation. Quantification of pathogen recovered from biofilm after daily challenges to control and coated (Coating C) PICC line segments by MRSA (FIG. 4A), P. aeruginosa (FIG. 4B), K. pneumoniae (FIG. 4C), and C. albicans (FIG. 4D). Samples were challenged with fresh inocula and media daily, and triplicates were removed for quantification on indicated days. Error bars represent standard deviation. * p<05. (e-f) SEM imaging of biofilm prevention.

FIG. 4E: Washed, uncoated PICC line surface after seven days of challenge with P. aeruginosa. FIG. 4F: Coated PICC line surface after seven days of challenge with P. aeruginosa.

FIGS. 5A-5D: Efficacy of coated (Coating C) PICC line segments in a high-protein (70 mg/mL serum protein) environment. Quantification of planktonic pathogen after daily challenges to control and coated PICC line segments in FBS-supplemented growth media. Pathogens quantified are MRSA (FIG. 5A), P. aeruginosa (FIG. 5B), K. pneumoniae (FIG. 5C), and C. albicans (FIG. 5D). Error bars represent standard deviation. * p<. 05.

### DETAILED DESCRIPTION

### I. Introduction

Given the severity of multi-drug resistant (MDR) infections in healthcare outcomes, efforts have been made to obtain novel antibiotics to combat current resistance patterns, and an attractive starting point is consideration of endogenous mechanisms for controlling microbial growth. Cationic antimicrobial peptides (AMPs) are one such natural source of pathogen control, utilizing electrostatic and hydrophobic interactions to permeabilize membranes and allow leakage of intracellular components of bacteria and fungi. These AMPs are ubiquitous in multicellular eukaryotes, and yet they remain effective at controlling invasive pathogens, indicating that their mechanisms are unlikely to induce strong mechanisms of resistance. Investigation of these peptides as therapeutics has shown promise, but there are substantial obstacles to their application, such as difficulty of large-scale synthesis, hydrolytic degradation, rapid system clearance, and thermal instability.

Cationic steroidal antimicrobial (“CSA”) compounds are a class of small molecule, non-peptide molecules that have more recently been found to exhibit amphipathic and membrane-disrupting mechanisms of AMP's while avoiding many of the obstacles of utilizing AMPs. The terms “ceragenin” and cationic steroidal antimicrobial” (or “CSA”) are used interchangeably herein. Ceragenins such as CSA-131 can be utilized against MDR strains such as Klebsiella pneumonia and Pseudomonas aeruginosa. Beneficially, tested ceragenins such as CSA-131 do not suffer from cross resistance with other membrane-active antimicrobials, such as chlorhexidine and colistin.

Due to the efficacy against MDR strains of high concern, an attractive application of ceragenins may be found in preventing colonization of medical devices. The relatively low cost of production of ceragenins, such as CSA-131, and their stability offer the possibility of incorporating them into manufacturing/distribution processes which require high temperatures and/or long storage times, which leverage their substantial advantages over AMPs and other antimicrobials.

Multiple approaches have been explored to inhibit the microbial colonization of medical devices such as catheters. Conventional approaches, in general, employ multiple antimicrobials/antibiotics to provide a spectrum of activity that encompasses Gram-positive and Gram-negative organisms. These combinations typically include antibiotics to which resistant bacteria have been identified. Due to the breadth of the spectrum of ceragenins, they may be used as a single type of antimicrobial to prevent colonization by Gram-positive and Gram-negative bacteria and fungi. Furthermore, as a mimic of AMPs, ceragenins are unlikely to engender resistance. Consequently, ceragenin-containing coatings are well suited as stand-alone, long-term solutions to the microbial colonization of medical devices such as catheters.

### II. Overview of CSA Compounds

CSA compounds, also referred to as “CSAs”, “CSA molecules”, or “ceragenin” compounds, are synthetically produced, small molecule chemical compounds that include a sterol backbone having various charged groups (e.g., amine, guanidine, and/or other cationic groups capable of exhibiting cationic properties under biological conditions) attached to the backbone. The sterol backbone can be used to orient the cationic groups on a face or plane of the sterol backbone. CSAs are cationic and amphiphilic, based upon the functional groups attached to the backbone. They are facially amphiphilic with a hydrophobic face and a polycationic face.

Example CSA compounds can have a structure of Formula I with appropriate substituents (R groups).

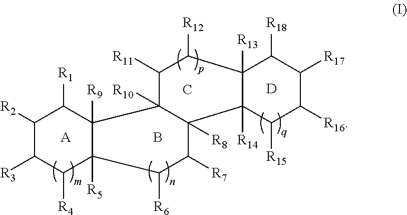

The CSA compounds can more particularly have a structure of Formula II, Formula III, or Formula IV:

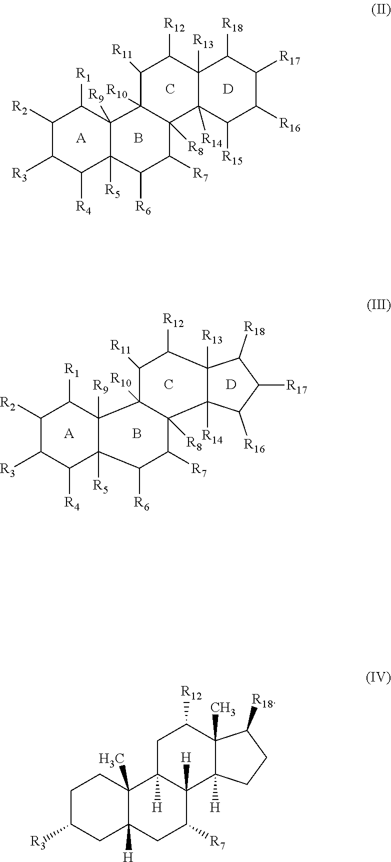

As will be discussed in greater detail below, the R groups of Formulae I, II, III and IV can have a variety of different functionalities, thus providing a given CSA compound with specific properties. The sterol backbone can be formed of 5-member and/or 6-member rings, so that p, q, m, and n in Formula I may independently be 1 (providing a 6-member ring) or 0 (providing a 5-member ring). Definitions for the R groups are set forth below.

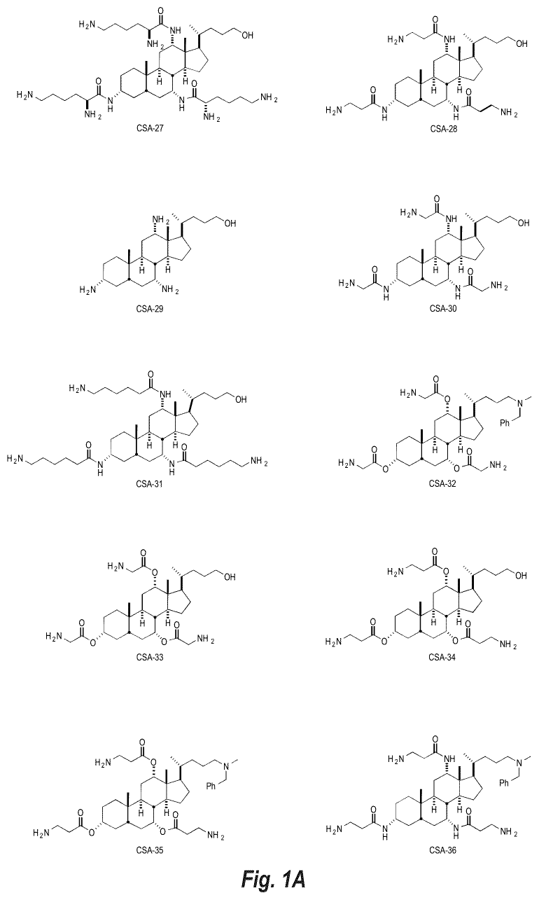

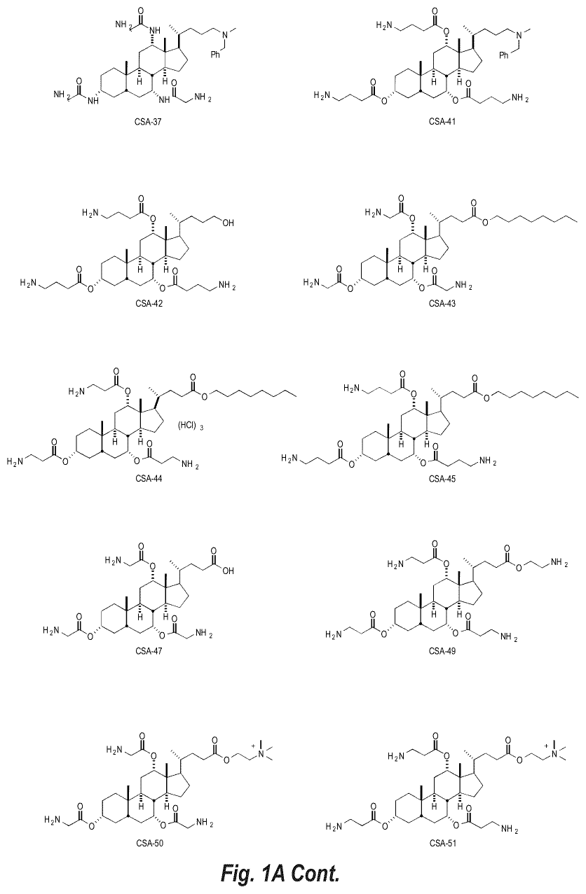

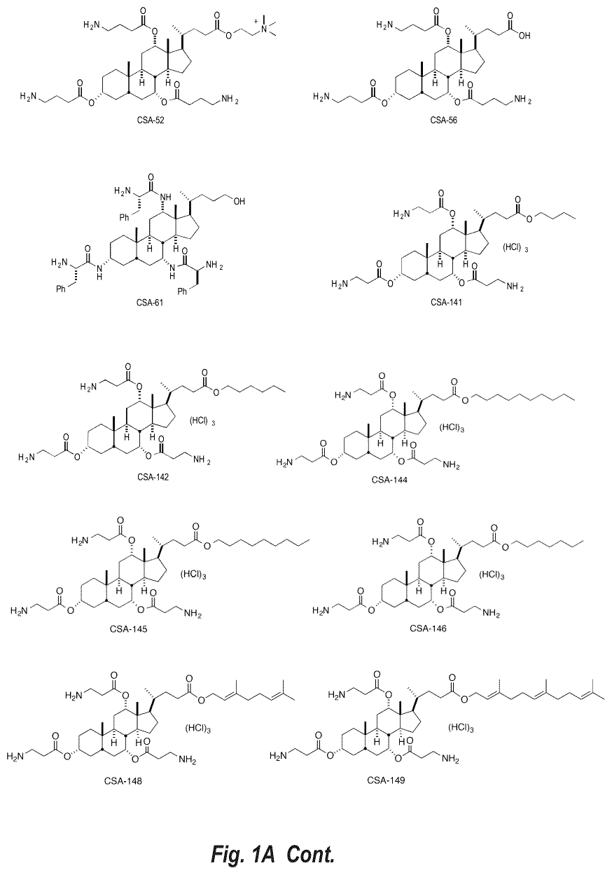

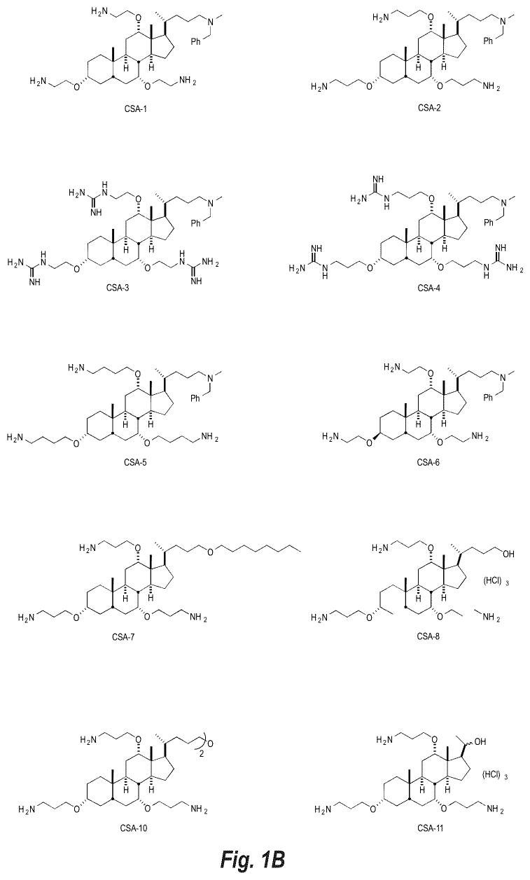

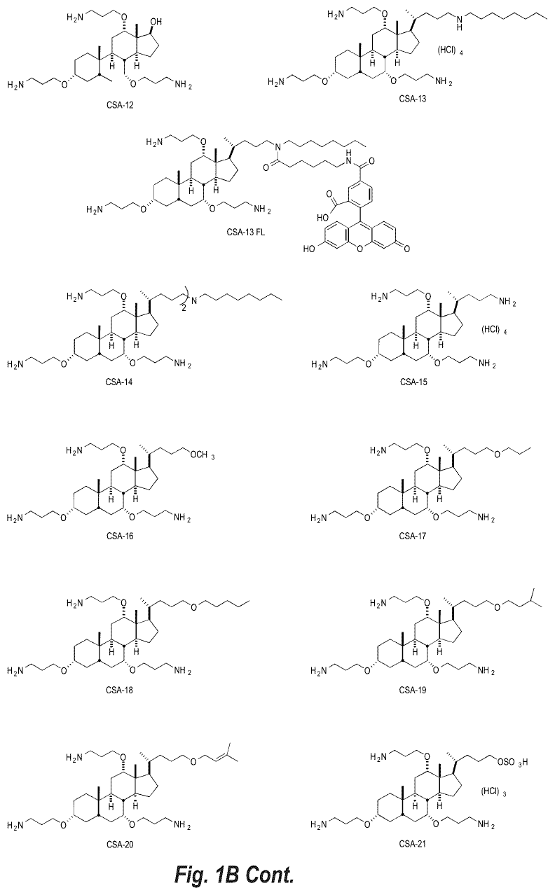

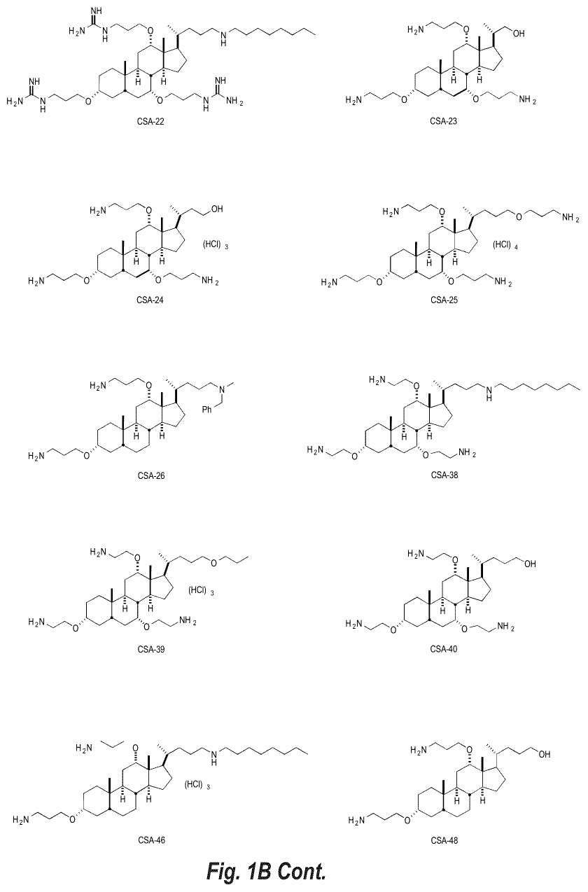

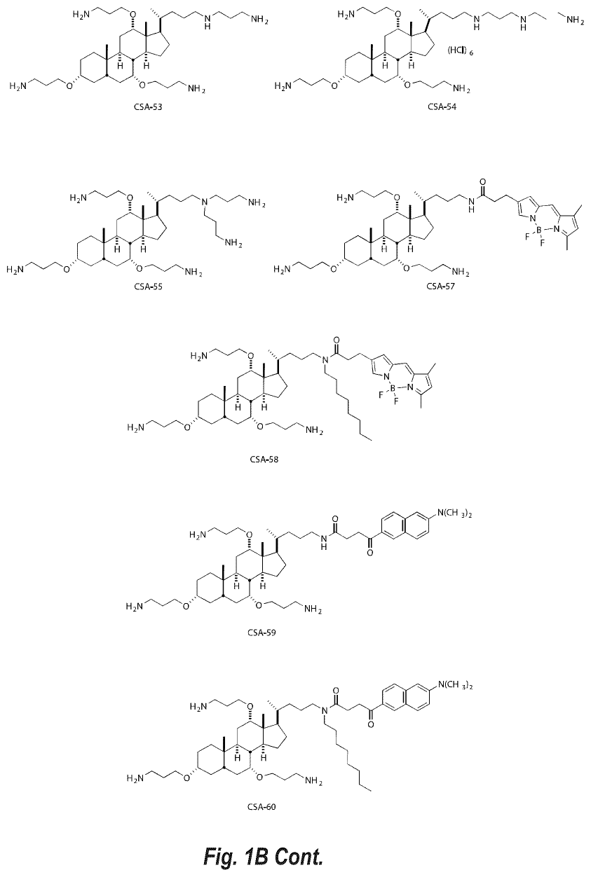

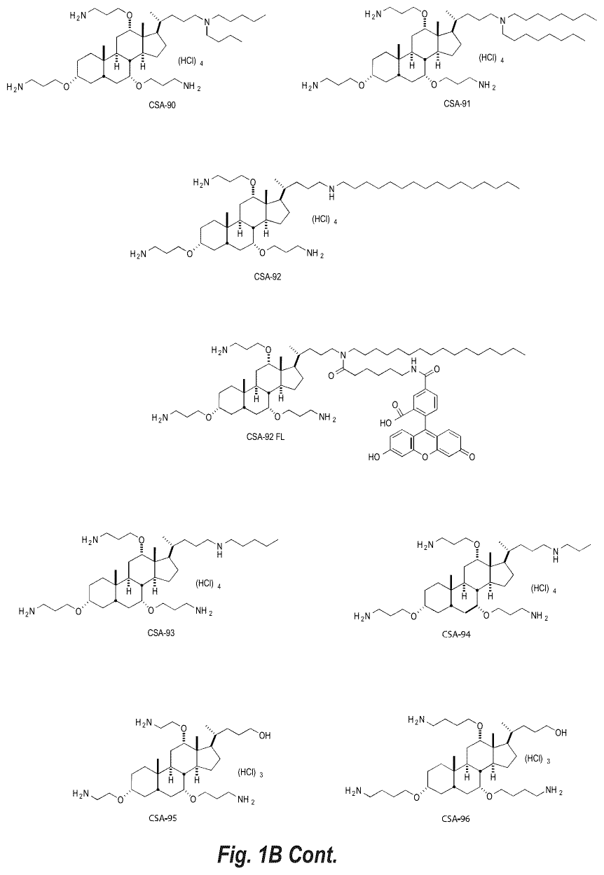

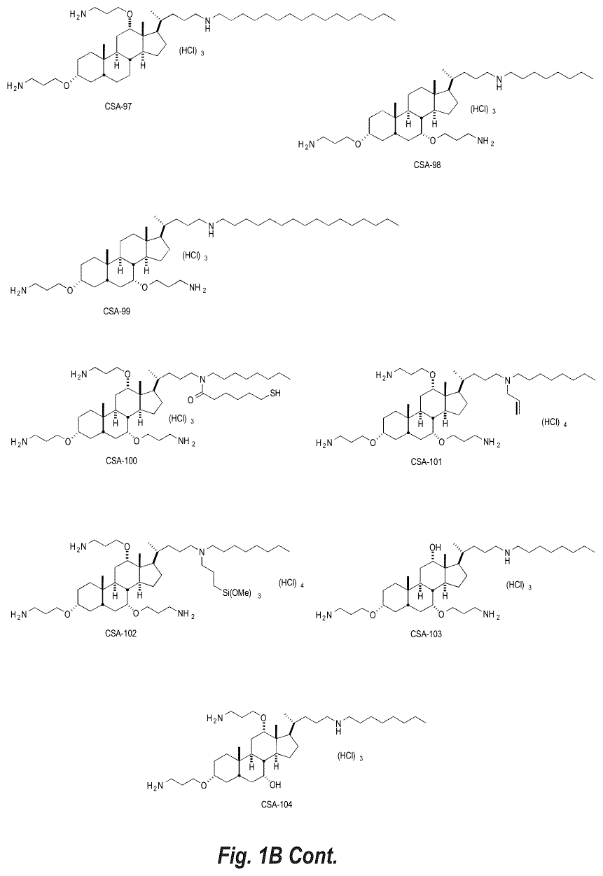

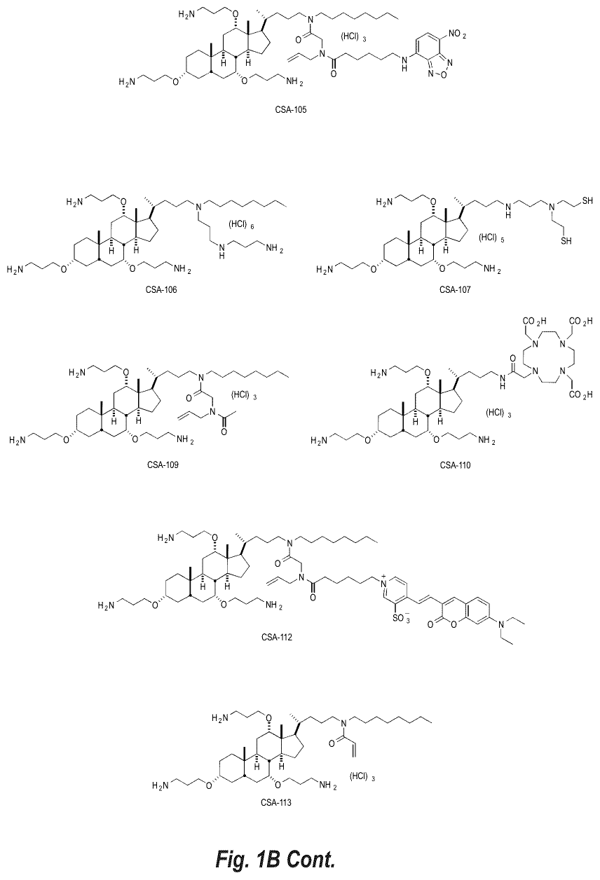

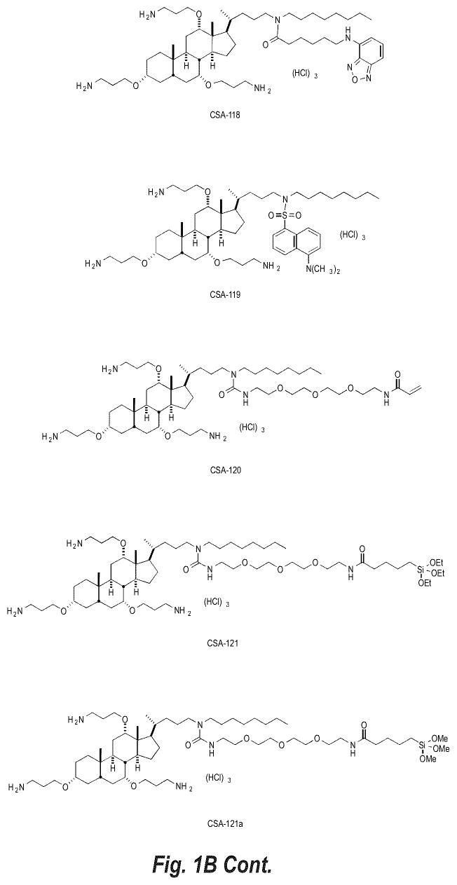

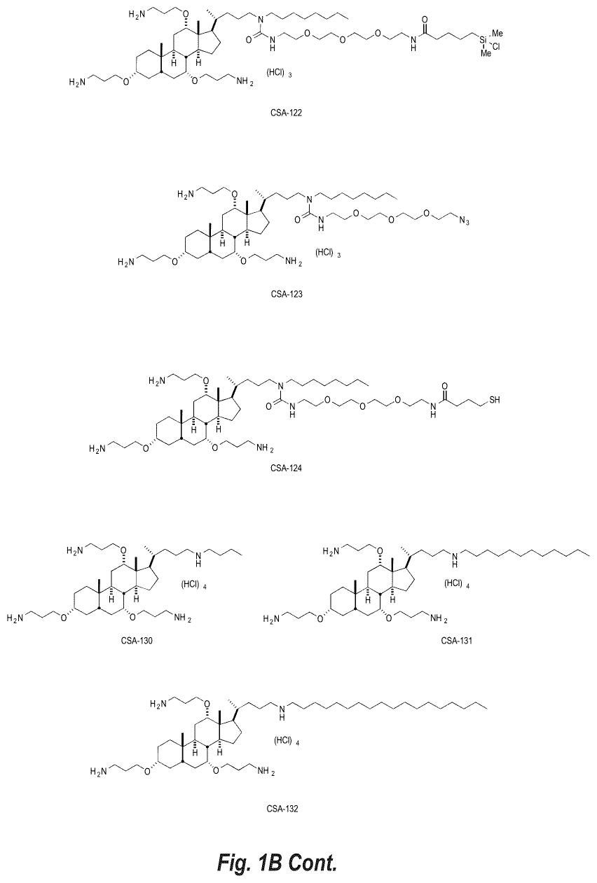

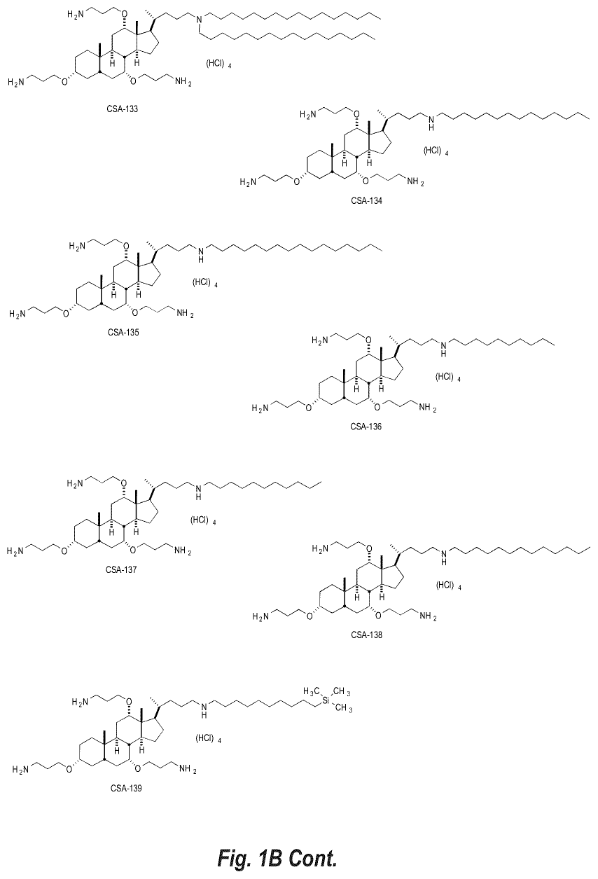

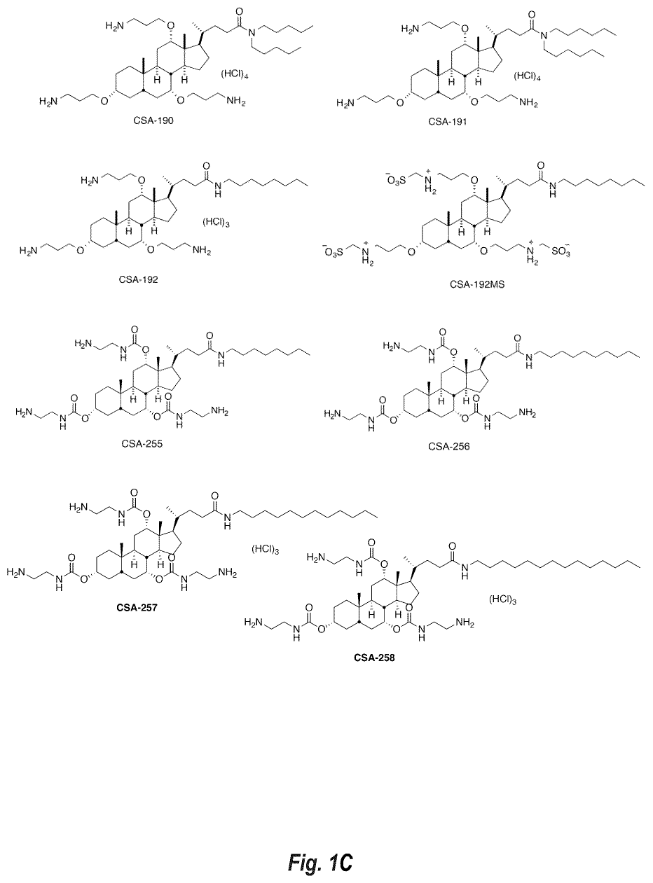

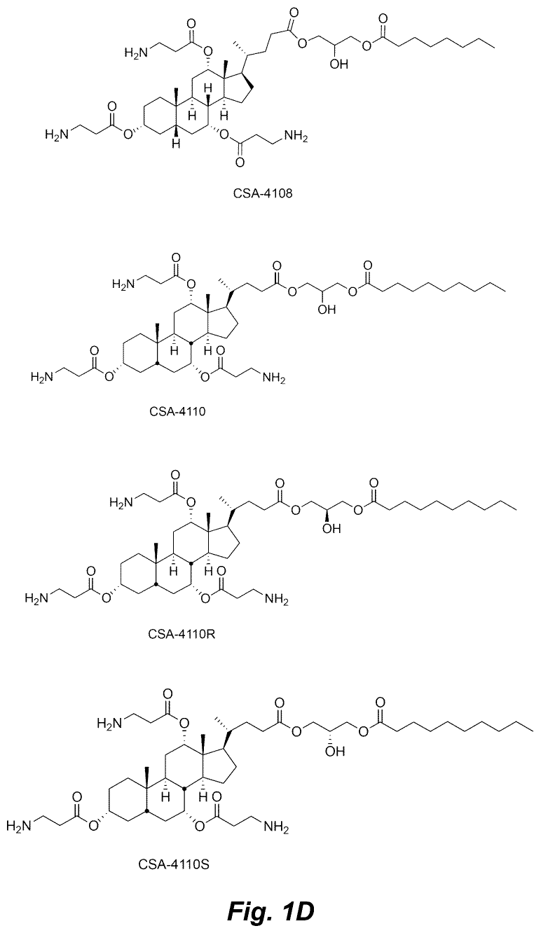

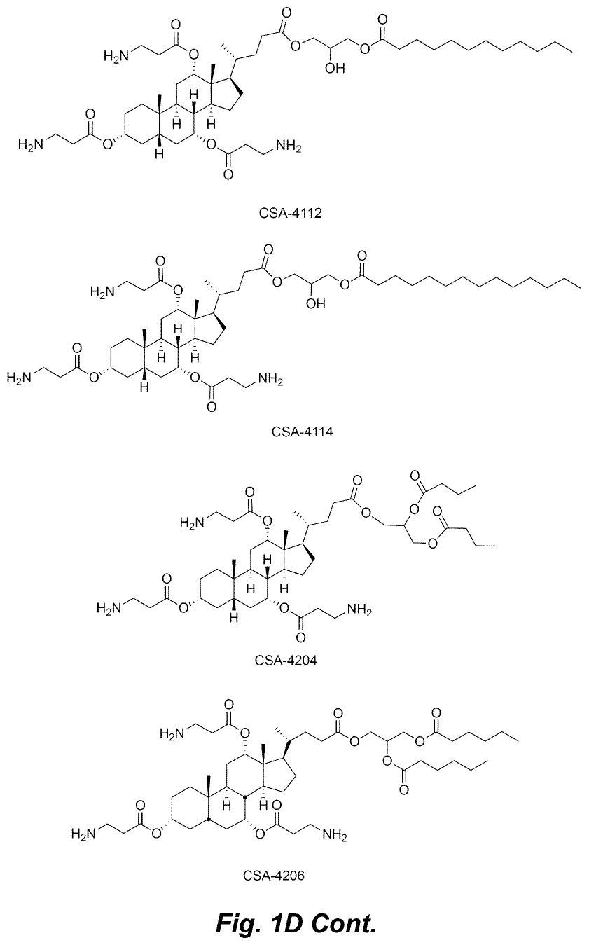

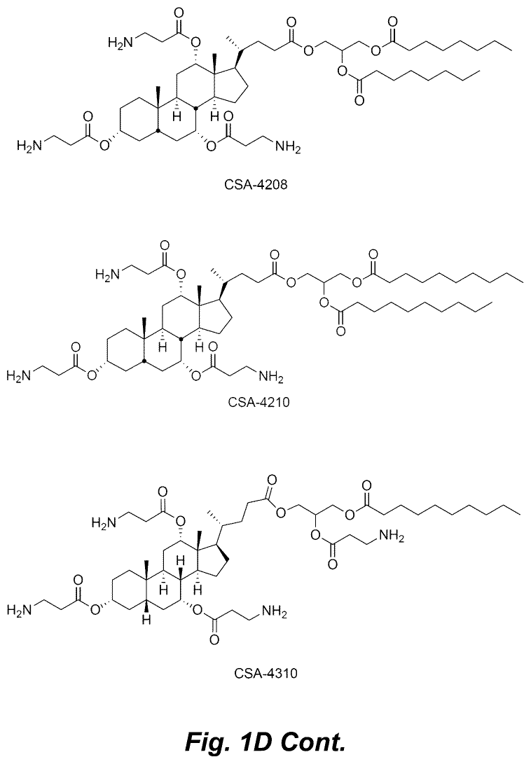

Formula II is a subset of Formula I in which rings A, B, C, and D are 6-member rings. Formula III is a subset of Formula I in which rings A, B, and C are 6-member rings and D is a 5-member ring. Formula IV is a subset of Formula III in which the stereochemistry is defined and the R groups other than R₃, R₇, R₁₂, and R₁₈ are defined as either hydrogen or methyl. Typically, the A, B, and C rings are 6-member rings and the D ring is a 5-member ring (e.g., Formulae III and IV). Examples of CSA compounds of Formula I, Formula II, Formula III, and Formula IV are illustrated in FIGS. 1A-1D.

Typically, CSAs used herein are of two types: (1) CSAs having cationic groups linked to the sterol backbone with hydrolysable linkages and (2) CSAs having cationic groups linked to the sterol backbone with non-hydrolysable linkages. For example, one type of hydrolysable linkage is an ester linkage, and one type of non-hydrolysable linkage is an ether linkage. CSAs of the first type can be “inactivated” by hydrolysis of the linkages coupling the cationic groups to the sterol backbone, whereas CSAs of the second type are more resistant to degradation and inactivation.

In preferred embodiments, CSA compounds of Formula I, Formula II, Formula III, and Formula IV are characterized by at least two of R₃, R₇, or R₁₂ independently including a cationic moiety attached to the sterol backbone via hydrolysable (e.g., ester) or non-hydrolysable (e.g., ether) linkages. A tail moiety is usually attached to Formula I at R₁₈. The tail moiety may be charged, uncharged, polar, non-polar, hydrophobic, or amphipathic, for example, and can thereby be selected to adjust the properties of the CSA and/or to provide desired characteristics. In a particularly preferred embodiment of a CSA compound, at least two of R₃, R₇, or R₁₂ include a cationic moiety attached to the sterol backbone via non-hydrolysable (e.g., ether) linkages, such CSA-131.

The activity of the CSA compounds can be affected by the orientation of the substituent groups attached to the backbone structure. In one embodiment, the substituent groups attached to the backbone structure are oriented on a single face of the CSA compound. Accordingly, each of R₃, R₇, and R₁₂ may be positioned on a single face of Formula I, Formula II, Formula III, and Formula IV. In addition, R₁₈ may also be positioned on the same single face.

### III. Coating Layer Incorporating CSA Compounds

The process of effectively incorporating an antimicrobial agent into a coating can be complicated by several factors, including chemical reactivity of the antimicrobial with the coating material, poor adherence of the coating to the substrate, and rapid loss of the antimicrobial from the coating.

For example, CSA compounds such as CSA-131 contain multiple amine groups, which can be reactive with monomers and prepolymers from which polymeric coatings are generated. Furthermore, these amine groups can make CSA compounds highly water-soluble, which promotes rapid elution from a coating.

To minimize these issues, CSA compounds can be included in the form of a sulfonic acid addition salt, such as a sulfonic acid addition salt wherein one or more sulfonic acid moieties are bonded to an aromatic moiety, such as a naphthalene sulfonic acid addition salt, such as a naphthalenedisulfonic acid (NDSA) salt, such as a 1,5-NDSA salt, preferably a di-addition salt of the CSA compound.

Such salt forms were found to be sparingly soluble in water, and ion exchange is required to allow the CSA compound to become more water soluble. The salt exchange process can control the release of the CSA compound from a coating.

Moreover, with such salt forms, the amine groups of the CSA compound are not reactive, thus preventing them from interacting with polymerization reactions. Such salt forms thus provide favorable attributes for incorporation into a polymeric coating for medical devices.

The CSA coating can be applied to a medical device via any suitable polymer application method. Examples include dip coating, spray coating, spin coating, electrospinning, electrophoretic deposition (EPD), solvent casting, thermal spraying, or combination thereof. A presently preferred method includes dip coating, and effective results have been demonstrated through this method, though other application methods are also expected to be effective.

One or more CSA compounds may be mixed with other components of a coating composition prior to or during application of the coating composition to the medical device. For example, a coating composition can be formulated to include one or more CSA compounds and suitable monomers and/or prepolymers which, when applied to the medical device, can cure to form the CSA coating layer. Example monomers and/or prepolymers can include those formulated to form a hydrophilic polymer, such as a hydrogel. Example monomers and/or prepolymers can include those formulated to form a polyurethane, silicone elastomer, polyethylene glycol (PEG), polyvinyl alcohol (PVA), fluoropolymer (e.g., polytetrafluorocthylenc), polyvinylpyrrolidone (PVP), polysulfone (PSU), or combination thereof.

The solvent(s) in which the monomers and/or prepolymers are mixed can be varied. Example solvents include water and organic solvents such as ketones (e.g., methyl ethyl ketone, acetone), alcohols (e.g., isopropyl alcohol, ethanol, butanol), esters (e.g., ethyl acetate, n-butyl acetate, and other esters formed between carboxylic acids and alkanols), carbonate-based solvents (e.g., dimethyl carbonate), aliphatic hydrocarbons, aromatic hydrocarbons, ethers, halogenated hydrocarbons, other suitable solvents, and combinations thereof.

The total amount of monomers/prepolymers can be included in the CSA coating composition at a concentration of 2% (w/v) to 10% (w/v), or 3% (w/v) to 8% (w/v), or 4% (w/v) to 7% (w/v), or 5% (w/v), or within a range with endpoints defined by any two of the foregoing values, based on total volume of the CSA coating composition.

The one or more CSA compounds can be included in the CSA coating composition at a concentration of 10% (w/w) to 60% (w/w), or 20% (w/w) to 50% (w/w), or 30% (w/w) to 40% (w/w), or within a range with endpoints defined by any two of the foregoing values, based on weight of total solids of the CSA coating composition.

Effective results have been demonstrated using a concentration of about 20% (w/w) of total solids. Higher concentrations, such as about 50% (w/w) of total solids, can also provide effective results while allowing significantly more loading of the one or more CSA compounds into the CSA coating. However, at such higher concentrations, the CSA coating was found to excessively elute loaded CSA compounds during the initial period following exposure to physiological conditions (e.g., during the first 24 hours). Such concentrations were also associated with excessive swelling of the CSA coating layer upon exposure to physiological conditions, which is likely due to insufficient crosslinking of binder components.

A multi-layered configuration, described in more detail below, was found to limit excessive early elution and to enable a more sustained release profile even at such higher CSA concentrations.

A primer layer can optionally be applied to the base substrate of the implantable medical device prior to application of the CSA coating layer. The primer layer can include, for example, primers based on silicone, silane coupling agent, PEG, acrylic/acrylate (including methacrylic/methacrylate forms and alkyl ester derivatives), epoxy, polyurethane, other suitable primers, and combinations thereof. A primer layer can beneficially enhance uniform distribution of the subsequent CSA coating layer and can reduce variability in mass changes of the medical device associated with the coating application process.

### IV. Multi-Layer Configurations

As described above, higher loading of CSA compound within the CSA coating layer can be beneficial; however, such high concentrations were found to excessively elute from the cured CSA coating layer once the CSA coating layer was exposed to physiological conditions. CSA coating layers with such loading rates also exhibited excessive swelling once exposed to physiological conditions.

Beneficially, including a top coating layer disposed over the CSA coating layer was found to limit these drawbacks while still allowing for the relatively high loading rates of CSA compound(s) within the CSA coating layer. That is, the top coating layer promotes more sustained elution of the one or more CSA compounds from the multi-layered coating when exposed to a physiological environment, as compared to the otherwise same coating that does not include the top coating layer.

The top coating layer can include any suitable polymer, such as any of the example polymers disclosed herein for the CSA coating layer. The top coating layer can be formed from a top coating composition that includes a monomer/prepolymer concentration such as those examples disclosed herein for the CSA coating layer.

In some embodiments, the top coating layer is formed of the same polymer or combination of polymers as the CSA coating layer (but omits the one or more CSA compounds). In other embodiments, the top coating layer has a polymer formulation different from the CSA coating layer. In presently preferred embodiments, the top coating layer has the same polymer formulation as the CSA coating layer but omits the one or more CSA compounds.

Although the ratio of the thickness of the CSA coating layer to the thickness of the top coating layer can vary, effective results were observed using a thickness ratio (CSA coating layer to top coating layer) of 0.75 to 3, such as 1 to 2.5, such as 1.25 to 2, such as 1.5, or a ratio that is within a range with endpoints defined by any two of the foregoing values. Thickness ratios within the foregoing ranges were associated with effective elution profiles and ability to maintain structural integrity of the multi-layer coating when exposed to a physiological environment.

### V. Antimicrobial Efficacy

The multi-layer coating disclosed herein is beneficially capable of providing antimicrobial effects against both planktonic and biofilm forms of microbial growth on an associated medical device.

For example, the multi-layer coating can provide multiple weeks (e.g., one week, two weeks, three weeks, or more than three weeks) of at least a 50% reduction (or at least a 1-log reduction, 2-log reduction, or 3-log reduction) in planktonic microbial growth on the medical device as compared to the same medical device without a coating layer that includes one or more CSA compounds. These antimicrobial effects can be exhibited against pathogens of critical or high concern, including, for example, methicillin-resistant S. aureus (MRSA), P. aeruginosa, K. pneumoniae, and C. albicans.

In another example, the multi-layer coating can provide a reduction in biofilm growth on the implantable medical device, as compared to the same medical device without a coating layer that includes one or more CSA compounds, for multiple weeks (e.g., one week, two weeks, three weeks, or more than three weeks). The relative reduction in biofilm growth can be at least 50%, at least 1-log reduction, at least 2-log reduction, or at least 3-log reduction, for example. Antimicrobial/antibiofilm effects can be exhibited against pathogens such as MRSA, P. aeruginosa, K. pneumoniae, and C. albicans, for example.

### VI. Working Examples

### 1. Materials & Methods

### Preparation of PICC Line Segments:

Single lumen BARD PowerPICC™ 4F PICC lines (Lucent Medical Systems, USA) were used. Lines were cut into 15 mm sections and cauterized to isolate a single accessible side for preparation and testing. Segments were mounted on 22 gauge needles to facilitate handling before being primed as advised by the manufacturer (Hydromer 2314-172) and cured for 8 hours at 70° C. Urethane prepolymer solution (Hydromer 2018-20M) was acquired at 3% (w/v) and was adjusted to target concentrations through solvent removal using a Buchi R-100 Rotavapor System. For coating layers with CSA-131 (NDSA salt), powder was weighed, and polyurethane solution added to acquire target CSA-131 (NDSA salt) percent of total solids, and the final suspension was sonicated at 0° C. for 4-8 hours to achieve complete dispersal of solids. PICC line segments were submerged in the suspension for 5 seconds and moved to an oven to cure overnight at 90° C.

### Microbial Cultures:

All samples were cultured in trypticase soy broth (TSB) for bacteria and Emmons modified sabouraud dextrose broth (EMSDB) for fungi. All pathogens tested are prepared by introducing single colonies from fresh culture plates into culture media and allowed to grow overnight on an orbital shaker at 30° C. Overnight cultures were washed and centrifuged three times in phosphate buffered saline (PBS) and resuspended in PBS. Optical density (OD) readings were acquired at 600 nm on a Genesys 30 spectrophotometer. Aliquots of bacterial cultures were diluted to 10⁶ CFU/mL in 10% TSB or 70% fetal bovine serum (FBS) with 10% TSB for high protein testing. Aliquots of fungal cultures were diluted to 10³ CFU/mL in 10% EMSDB or 70% fetal bovine serum (FBS) with 10% EMSDB for high protein testing. Rationale for using 70% FBS is that whole blood protein is estimated at 70 mg/mL and serum alone contains approximately 100 mg/mL protein.

### Extraction & Elution of CSA-131:

Extraction and elution assays were run in triplicate. To obtain total extraction of CSA-131, PICC line segments were submerged in a solution comprised of 80% isopropanol and 20% 1 N HCl and heated to 70° C. for 8 h. Segments were transferred to new vessels and incubated in additional extraction solution at room temperature. Subsequent extraction steps continued at room temperature until CSA-131 peaks were indetectable by mass spectrometry. Daily elution samples were acquired from PICC line segments by incubation in PBS at 37° C. PBS was exchanged daily at the same time and collected samples run on the same day. Deuterated reagents had previously been used to synthesize CSA-131D25 which served as an internal standard for quantification by mass spectrometry (Agilent Technologies 6230 TOF LC/MS).

Pathogenic Challenge with Quantification of Planktonic Growth:

PICC line segments were placed in culture tubes and immersed in 700 μL of inocula and incubated at 30° C. for 24 h for bacteria or 37° C. for 24 h for fungi. Microbial growth was measured by removing aliquots into Dey-Engley Neutralizing Broth (Sigma-Aldrich, USA). The resulting suspensions were diluted and spread on nutrient agar (TSA for bacteria and EMSDA for fungi). Plates were incubated for 24 h before colonies were counted. Tests were run until statistical significance was lost (p<. 05).

Coatings cross-section preparation:

The cross-section of coatings was studied using scanning electron microscope Apreo C (Thermo Fisher, USA). Standard methods for preparing cross-section (polishing, cutting) are not suitable for studying coatings (especially multilayer ones) obtained on soft polymer samples, which is associated with the transfer of particles between layers and, as a result, blurring of the boundaries between them. In connection with the foregoing, the following methodology was used in this work. At the first stage, the coated samples were frozen by immersion in liquid nitrogen (from −195 to −200° C.) and exposure for at least 15 min. After this procedure, the fragility of the sample increased significantly, and the frozen samples were cracked by bending.

### Sample Preparation for Surface Study by Scanning Electron Microscopy:

PICC line segments were prepared as described above. Segments designated for the biofilm challenge were incubated for seven days as described above. After incubation, segments were washed with Sorensen buffer (0.1 M, pH 7.2) then fixed in 2.5% (w/v) glutaraldehyde in Sorensen buffer at 4° C. overnight, rinsed with Sorensen buffer, immersed in an osmium tetroxide solution (0.5 mL) for 2-3 h and washed with Sorensen buffer to remove excess osmium tetroxide. An ethanol series from 10% to 100% and hexamethyldisilazane were used to dehydrate the samples, which were then placed in a critical point drier (Tousimis 931 Autosamdri, USA) overnight. Samples were sputter-coated with approximately 20 nm of a gold-palladium alloy (Quorum Q 150T ES, Electron Microscopy Science, USA), and surfaces of PICC line segments were visualized under an Apreo C microscope (Thermo Fisher, USA)

### Quantification of Biofilm Growth:

For each pathogen, 30 coated and 30 control PICC line segments were randomly divided into 10 triplicates of each type, which were inoculated with cultures of the indicated pathogens. Every 24 h the existing growth medium was removed, the devices were rinsed three times with 1 mL of PBS, transferred to clean culture tubes and reinoculated in fresh growth medium. Samples were removed at predetermined intervals until significant growth was observed, defined as 15 colonies/plate, at which point samples were removed daily. Biofilm growth was quantified by removing a control and coated triplicate which were rinsed twice with PBS. Selected segments were transferred to a culture tube and rinsed a final time to remove planktonic cells. Neutralizing broth (1 mL) was added to the tubes, which were moved to a bath sonicator (Fisher Scientific FS60, 42 kHz 100 W) for 15 min, to disrupt biofilm. The segments were vortexed before samples were taken, serially diluted, and plated on agar (TSA for bacteria, EMSDA for fungi). Plates were incubated for 24 h and colonies were counted.

### 2. Results

CSA-131 (NDSA Salt) can be Stably Integrated into Polyurethane Coatings:

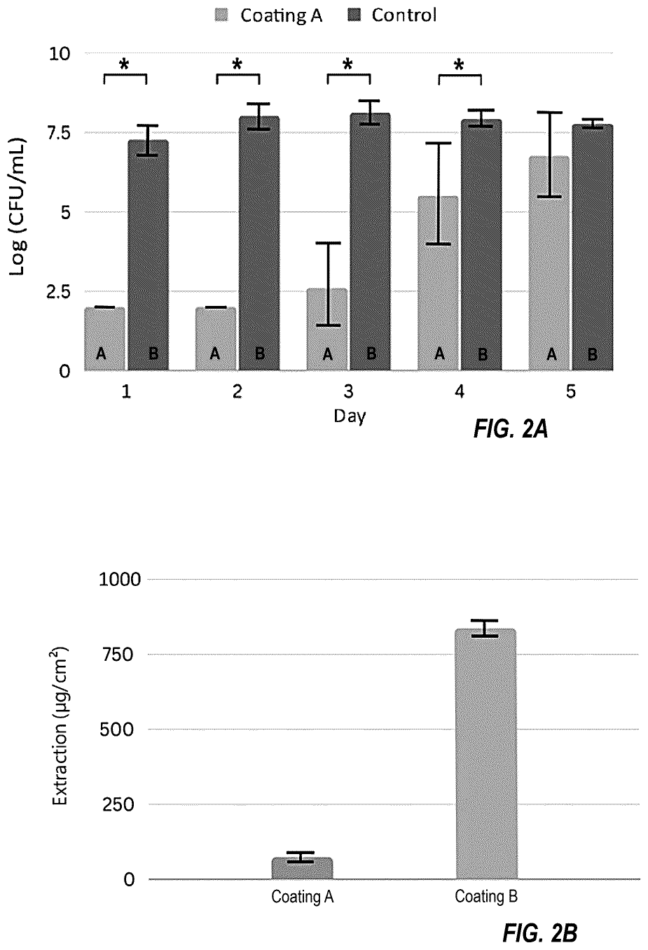

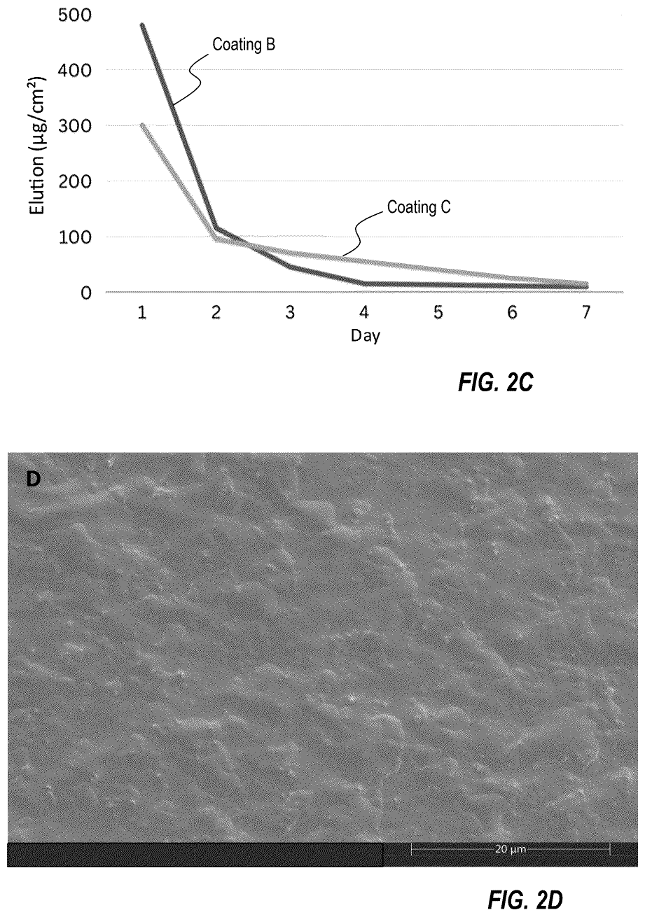

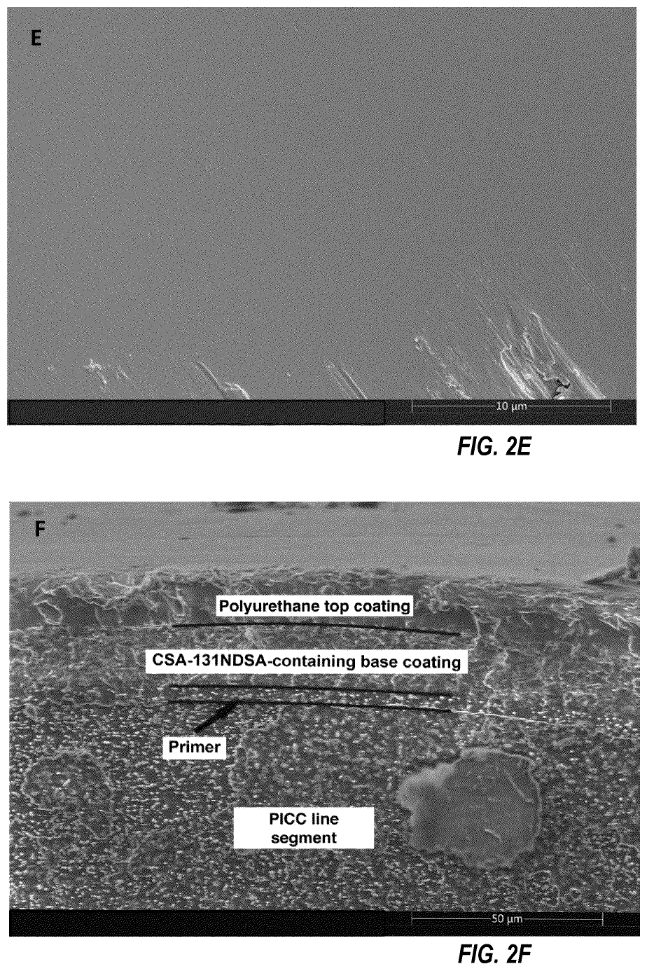

It was determined that utilizing a 3% (w/v) urethane prepolymer solution in methyl ethyl ketone resulted in a layer of material which would adhere to the surface in both dry and wet condition. It was also determined that the inclusion of an initial silicone primer on the PICC line segments reduced variability in mass changes associated with the dip-coating process, and all samples were subsequently primed. By suspending CSA-131 (NDSA salt) at 20% (w/w) of total solids in the urethane solution, the first coating system was attained (Coating A). Initial results provided a 3-log reduction in colony forming units (CFU) for three days with daily challenges by methicillin-resistant Staphylococcus aureus (MRSA) (FIG. 2A).

### Multi-Layer Coatings can Control Total CSA-131 and Elution:

A second system contained 5% (w/v) urethane prepolymers with CSA-131NDSA at 50% (w/w) of total solids (Coating B). The increased concentrations and viscosity resulted in a nearly 12-fold increase in CSA-131 in the coating compared to the earlier system (FIG. 2B). The elution profile for this coating, however, indicated it was losing over half of the load of CSA-131 within 24 hours of exposure to an aqueous environment and visible swelling of the coating was observed, indicating that the ratio of solids in the coating did not allow sufficient crosslinking between urethanes for stability (FIG. 2C). Aiming to conserve the higher reservoir of CSA-131, reduce initial elution and increase stability, use of a top coating layer was investigated. After curing the initial CSA coating layer, segments were immersed in the same 5% urethane prepolymer solution that had been previously made but without CSA-131. After curing, the layered coatings led to a system (Coating C) that had no visible swelling in water, had a lower initial release of CSA-131, and a more sustained release on subsequent days (FIG. 2C).

### Polyurethane Coating Provides Distinct Layers Fully Covering the PICC Line Surface:

Scanning electron microscopy (SEM) was utilized to visualize Coating C, and initial observations of the surface of coated and uncoated PICC line segments verified that the coating left no exposed tubing (FIGS. 2D-2E). A freeze-crack method was used to expose the layers of the combined coating. Analysis of the cross-section of the coating revealed a structure consisting of several distinct layers (FIG. 2F). The first layer, approximately 4 μm thick, directly adjacent to the substrate, is the silicone primer layer. The next layer, which is approximately 18 μm thick, is the base polyurethane layer containing CSA-131NDSA (“the CSA coating layer”). Note that there is a clear boundary between the primer layer and the CSA coating layer, due at least partly to the inclusion of CSA-131NDSA in the CSA coating layer. The top coating layer, which is approximately 12 μm thick, is also clearly visible on the SEM image. Comparison of this layer and the underlying CSA coating layer shows a greater amount of heterogeneity in the lower layer, which likely allows a greater amount of solvent infiltration. The homogeneity of the top layer was expected to restrict solvent penetration and delay CSA-131 elution.

### Coated PICC Lines Reduce Planktonic Growth of Bacterial and Fungal Pathogens:

To test the antimicrobial efficacy of Coating C, representative Gram-positive and Gram-negative bacterial and fungal strains were selected, specifically MRSA, P. aeruginosa (PA), K. pneumoniae (KP), and C. albicans (CA). These were selected as they are responsible for a majority of central line-associated bloodstream infections (CLABSI) identified in clinical studies. Further, all have been designated as high or critical priority pathogens by the WHO.

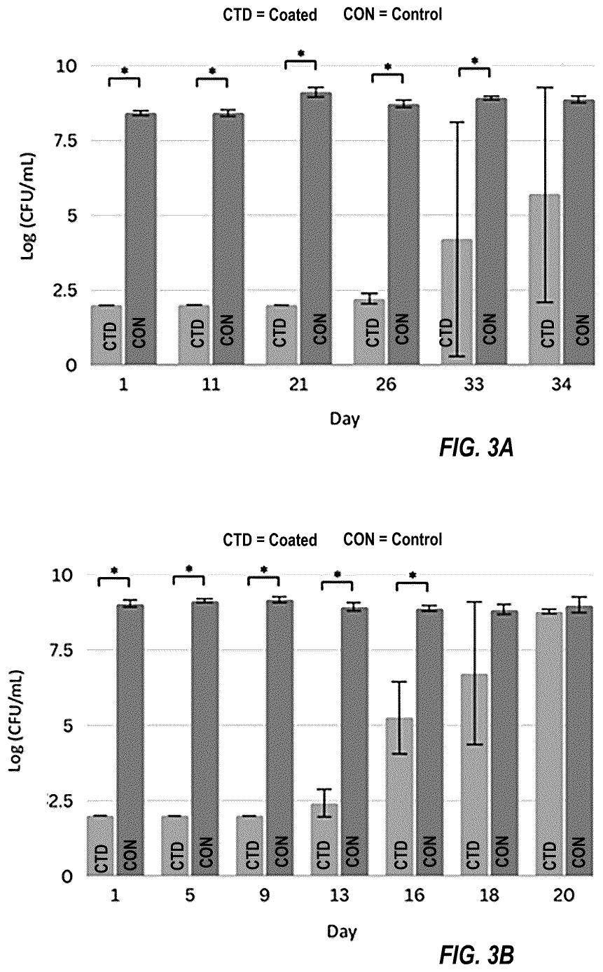

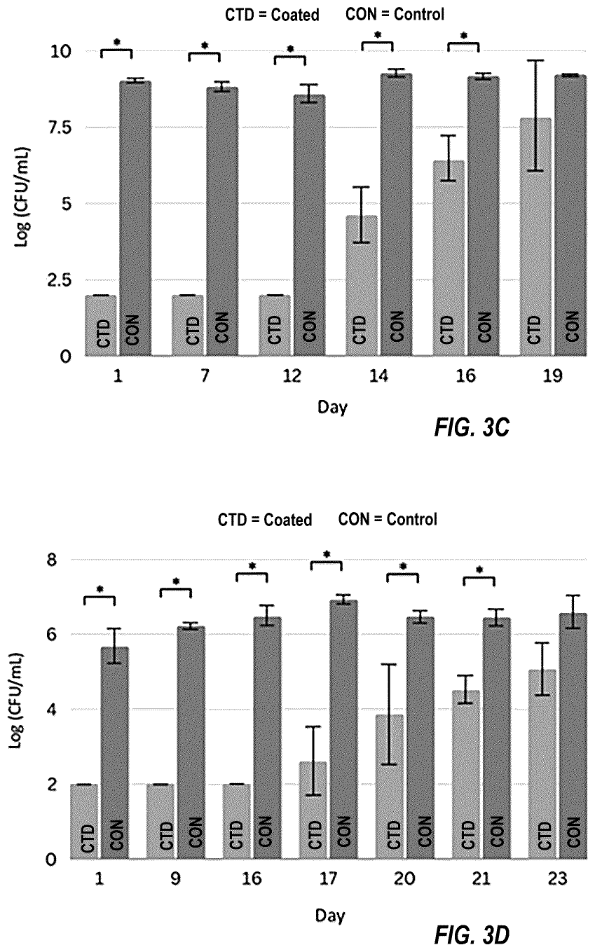

In this study, PICC line segments were exposed daily to inocula of the indicated pathogen in the appropriate fresh growth medium. It was found that PICC line segments with Coating C gave multiple weeks of at least a 3-log reduction in pathogenic growth. Against MRSA, the first growth in the medium appeared at day 26 and a total of 33 days of statistically significant reduction in bacterial growth was observed (FIG. 3A). With PA, growth was first detected at day 13 and 16 days of statistically significant reduction were observed (FIG. 3B). Coating C performed similarly with the other Gram-negative strain, KP, with first growth at 14 days, and statistical significance reduction through day 16 (FIG. 3C). For CA, growth was observed at day 17 and significant reduction through day 21 (FIG. 3D). While the significant reductions of growth from pathogens of concern for multiple weeks remains a promising benchmark, these assays have described coating activity against planktonic forms of the microorganisms; an even greater indicator of potential success is the prevention of biofilm formation.

### Coated PICC Lines Prevent Biofilm Formation:

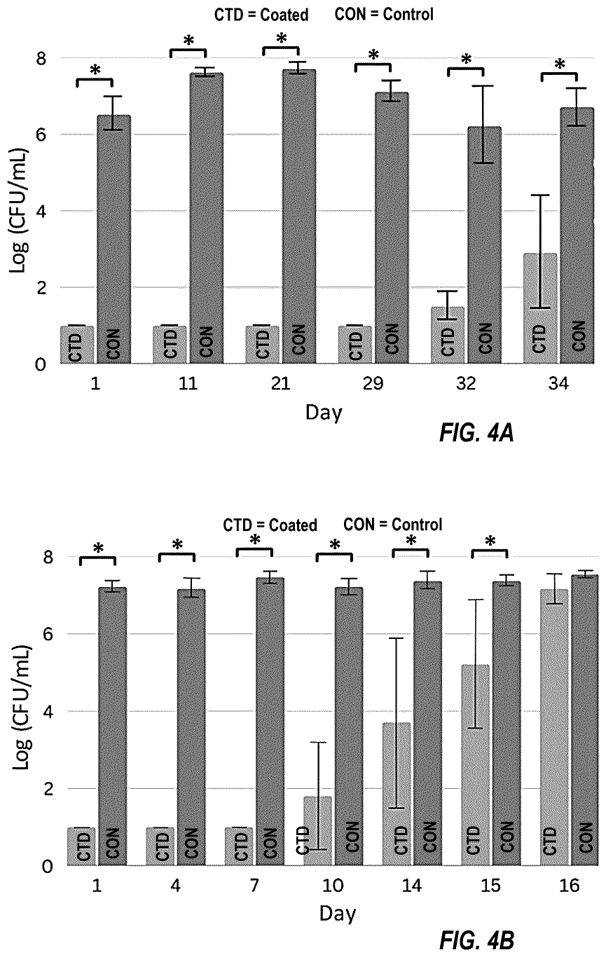

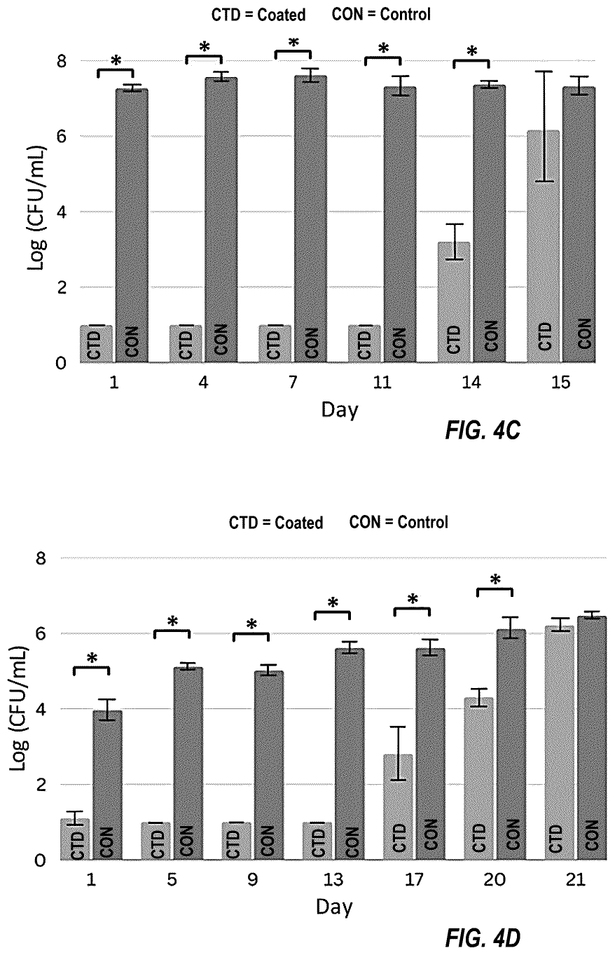

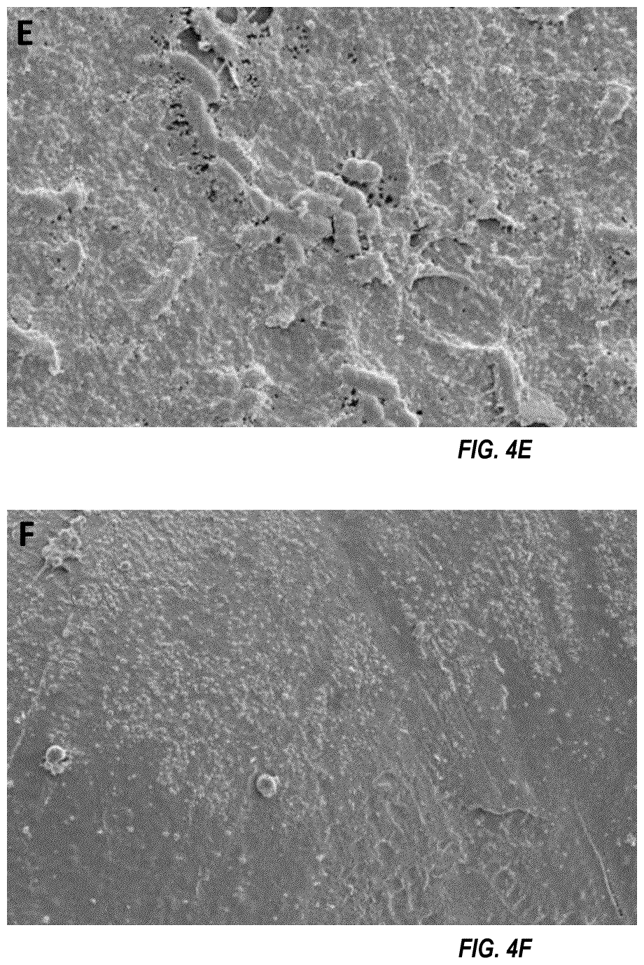

Coated (Coating C) and control PICC line segments were prepared and challenged daily with fresh inocula in the appropriate medium. With bacteria, counts within established biofilms on uncoated PICC line segments were 6-8 logs after one day of incubation (FIGS. 4A-4C), and with CA, biofilm counts were 4 to just over 6 logs (FIG. 4D). Counts remained in these ranges even after extensive reinoculation and incubation. Segments with Coating C were protected for extended periods from colonization. For example, with MRSA, after a single day of incubation, the number of bacterial counts on coated segments was below the detection limit (1 log), and counts remained at this level for 29 days of repeated inoculations and growth medium exchanges (FIG. 4A). By day 34, bacteria were isolated from the coated segments; however, as compared to control, the coating reduced the biofilm by more than 3 logs. With the other organisms, Coating C protected the segments for at least 14 days (FIGS. 4B-4D).

Whether biofilm formation was completely prevented or if some biofilm formation occurred and was eliminated within the sampling window was additionally monitored. In order to visualize biofilm on coated segments, SEM and samples challenged with PA were used. Comparing the unchallenged PICC line surface (FIG. 2D) and the segment challenged by PA for 7 days (FIG. 4E) demonstrates the ability of PA to establish a mature biofilm on the uncoated surface. A sheet of matrix proteins has been established and individual rod-shaped cells can be seen both under the surface and partially exposed, potentially representing cells about to be shed from the biofilm. Conversely, comparing the unchallenged coating (FIG. 2E) and the challenged coating (FIG. 4F) shows the surface free of established protein matrix and rod-shaped cells. Small aggregates can be observed on the surface which may represent cellular remnants of bacteria, but no evidence of biofilm formation was observed.

### Efficacy Against Pathogens in High-Protein Environments:

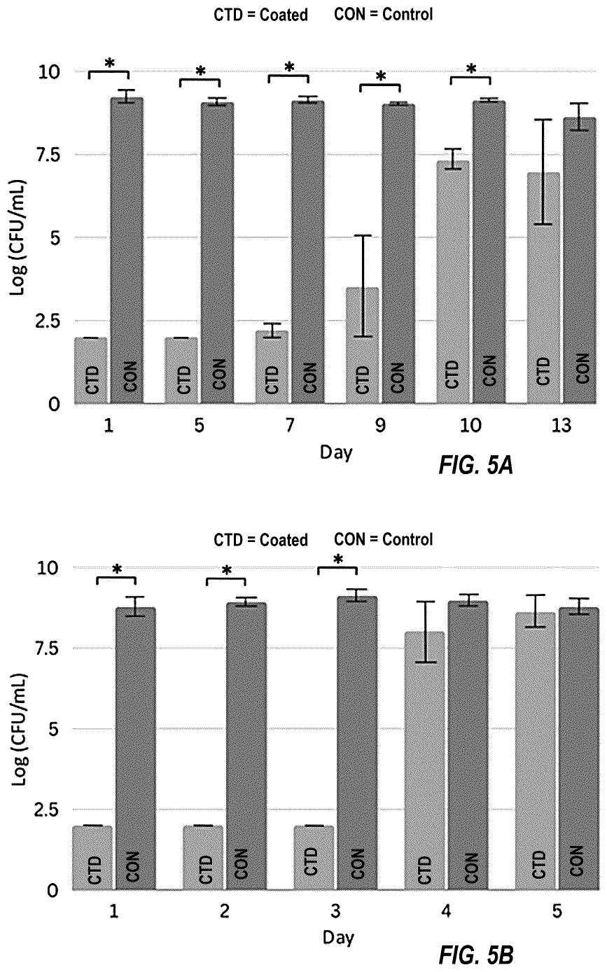

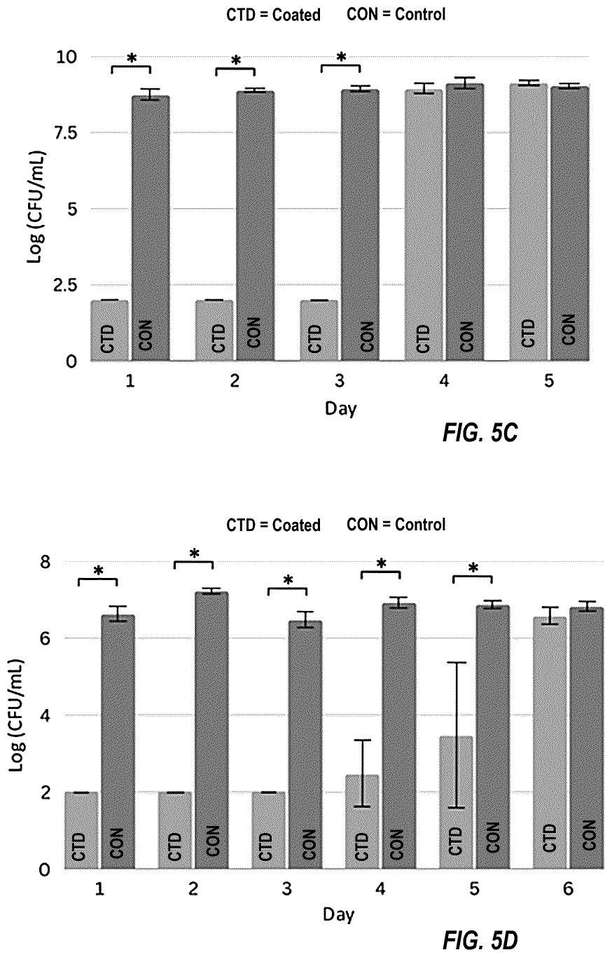

Implanted medical devices may be exposed to high protein concentrations, and protein deposition on surfaces can influence microbial adhesion. In addition, the antimicrobial efficacy of antibiotics can be influenced by high protein concentrations, and protein aggregation has been observed as a mechanism of antibiotic resistance. Therefore, the planktonic efficacy assays in a high protein environment were repeated. Media containing 70 mg/mL serum protein supplemented with standard growth media to challenge PICC line segments were prepared. In the presence of coated (Coating C) segments, MRSA growth was significantly reduced for 10 days (FIG. 5A), while with the Gram-negative pathogens, microbial growth was observed on Day 4 (FIG. 5B-5C). The coated segments significantly inhibited growth of CA for five days (FIG. 5D).

### VII. Additional Terms & Definitions

While certain embodiments of the present disclosure have been described in detail, with reference to specific configurations, parameters, components, elements, etcetera, the descriptions are illustrative and are not to be construed as limiting the scope of the claimed invention.

For any given element of component of a described embodiment, any of the possible alternatives listed for that element or component may generally be used individually or in combination with one another, unless implicitly or explicitly stated otherwise.

The various features of a given embodiment can be combined with and/or incorporated into other embodiments disclosed herein. Thus, disclosure of certain features relative to a specific embodiment of the present disclosure should not be construed as limiting application or inclusion of said features to the specific embodiment. Rather, it will be appreciated that other embodiments can also include such features.

Unless otherwise indicated, numbers expressing quantities, constituents, distances, or other measurements used in the specification and claims are to be understood as optionally being modified by the term “about.” When the terms “about,” “approximately,” “substantially,” or the like are used in conjunction with a stated amount, value, or condition, it may be taken to mean an amount, value or condition that deviates by less than 10%, less than 5%, less than 1%, less than 0.1%, or less than 0.01% of the stated amount, value, or condition. Each numerical parameter should be construed in light of the number of reported significant digits and by applying ordinary rounding techniques.

Any headings and subheadings used herein are for organizational purposes only and are not meant to be used to limit the scope of the description or the claims.

As used in this specification and the appended claims, the singular forms “a,” “an” and “the” do not exclude plural referents unless the context clearly dictates otherwise. Thus, for example, an embodiment referencing a singular referent (e.g., “CSA compound”) may also include two or more such referents.

The embodiments disclosed herein should be understood as comprising/including disclosed components, and may therefore include additional components not specifically described. Optionally, the embodiments disclosed herein are essentially free or completely free of components that are not specifically described. That is, non-disclosed components may optionally be completely omitted or essentially omitted from the disclosed embodiments. For example, polymers and/or coating components not disclosed herein may optionally be completely omitted or essentially omitted.

An embodiment that “essentially omits” or is “essentially free of” a component may include trace amounts and/or non-functional amounts of the component. For example, an “essentially omitted” component may be included in an amount no more than 1%, no more than 0.1%, or no more than 0.01% by total weight of the relevant composition (e.g. by total weight of the coating composition from which a coating layer is formed).

A composition that “completely omits” or is “completely free of” a component does not include a detectable amount of the component (i.e., does not include an amount above any inherent background signal associated with the testing instrument) when analyzed using standard compositional analysis techniques such as, for example, chromatographic techniques (e.g., thin-layer chromatography (TLC), gas chromatography (GC), liquid chromatography (LC)), or spectroscopy techniques (e.g., Fourier transform infrared (FTIR) spectroscopy).

### VIII. Additional CSA Compound Details

Exemplary CSA compounds and methods for their manufacture are described in U.S. Pat. Nos. 6,350,738, 6,486,148, 6,767,904, 7,598,234, 7,754,705, 8,691,252, 8,975,310, 9,434,759, 9,527,883, 9,943,614, 10,155,788, 10,227,376, 10,370,403, 10,626,139, and 11,286,276, U.S. Pat. Pub. Nos. 2016/0311850, 2021/0355155 and 2021/0363174, and U.S. patent application Ser. No. 18/823,369, which are incorporated herein by reference. The skilled artisan will recognize the compounds within the generic formulae set forth herein and understand their preparation in view of the references cited herein and the Examples contained therein.

CSA compounds can have a structure of Formula I, Formula II, Formula III, and/or Formula IV. Formula III differs from Formula I and II by omitting R₁₅ and the ring carbon to which it is attached. Formula IV more particularly defines Formula III with respect to stereochemistry and R groups for all but R₃, R₇, R₁₂, and R₁₈.

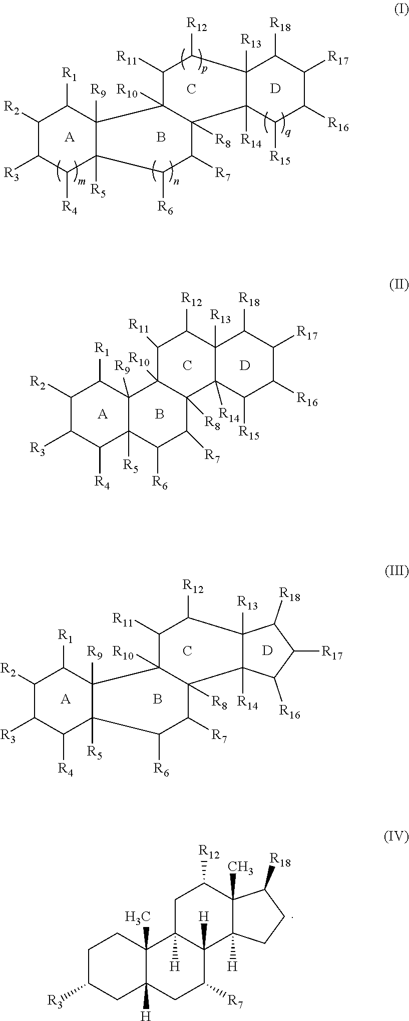

In embodiments of Formulas I, II, III, and IV, at least two of R₃, R₇, and R₁₂ may independently include a cationic moiety (e.g., amino or guanidino groups) bonded to the steroid backbone structure via a hydrolysable or non-hydrolysable linkage. For the embodiments of the present disclosure, the linkage is preferably hydrolysable but stable under conditions of sterilization and storage, and hydrolysable under physiological conditions. Such cationic functional groups (e.g., amino or guanidino groups) may be separated from the backbone by at least one, two, three, four or more atoms.

A tail moiety may be attached to the sterol backbone at R₁₈, may have variable chain length or size, and may be charged, uncharged, polar, non-polar, hydrophobic, or amphipathic. The tail moiety may be used to select the hydrophobicity/hydrophilicity of the ceragenin compound. CSA compounds having different degrees of hydrophobicity/hydrophilicity may have different rates of uptake into different target microbes.

The “R” groups described herein, unless specified otherwise, may be substituted or unsubstituted.

With respect to CSA compounds of Formulas I, II, and III (and where not already specified with respect to Formula IV):
each of fused rings A, B, C, and D may be independently saturated, or may be fully or partially unsaturated, provided that at least two of A, B, C, and D is saturated, wherein rings A, B, C, and D form a ring system. Other ring systems can also be used, e.g., 5-member fused rings and/or compounds with backbones having a combination of 5- and 6-membered rings;
R₁ through R₁₈ are independently selected from the group consisting of hydrogen, hydroxyl, alkyl, hydroxyalkyl, alkyloxyalkyl, alkylcarboxyalkyl, terpenylcarboxyalkyl, terpenylcarbonyloxyalkyl, terpenylamidoalkyl, terpenylaminoalkyl, terpenyloxyoalkyl, alkylaminoalkyl, alkylamino-alkylamino, alkylaminoalkylaminoalkylamino, aminoalkyl, aryl, arylaminoalkyl, haloalkyl, alkenyl, alkynyl, oxo, linking group attached to a second steroid, aminoalkylurethanyl, aminoalkenylurethanyl, aminoalkynylurethanyl, aminoarylurethanyl, aminoalkyloxy, aminoalkylcarboxy, aminoalkyloxyalkyl, aminoalkylaminocarbonyl, aminoalkylcarboxamido, di(alkyl)aminoalkyl, H₂N—HC(Q₅)—(C═O)—O—, H₂N—HC(Q₅)—(C═O)—NH—, azidoalkyloxy, cyanoalkyloxy, P.G.-HN—HC(Q₅)—(C═O)—O—, guanidinoalkyloxy, quaternary ammonium alkylcarboxy, and guanidinoalkyl carboxy, where Q₅ is a side chain of any amino acid (including a side chain of glycine, i.e., H), and P.G. is an amino protecting group; and
R₅, R₈, R₉, R₁₀, R₁₃, R₁₄ and R₁₇ are independently deleted when one of rings A, B, C, or D is unsaturated so as to complete the valency of the carbon atom at that site,
provided that at least one, and sometimes two, three, or four, of R₁₋₄, R₆, R₇, R₁₁, R₁₂, R₁₅, R₁₆, R₁₇, and R₁₈ are independently selected from the group consisting of aminoalkyl, aminoalkyloxy, aminoalkylcarboxyalkyl, alkylaminoalkyl, alkylamino-alkylamino, alkylaminoalkylaminoalkylamino, aminoalkylcarboxy, aryl-aminoalkyl, aminoalkyloxyamino, alkylaminocarbonyl, aminoalkylaminocarbonyl, aminoalkyl-carboxyamido, di(alkyl)aminoalkyl, aminoalkylurethanyl, aminoalkenyl-urethanyl, aminoalkynylurethanyl, aminoarylurethanyl, H₂N—HC(Q₅)—C(O)—O—, H₂N—HC(Q₅)—C(O)—N (H)—, azidoalkyloxy, cyanoalkyloxy, P.G.-HN—HC(Q₅)—C(O)—O—, guanidinoalkyloxy, quaternary ammonium alkylcarboxy, and guanidinoalkylcarboxy.

In embodiments, R₁ through R₄, R₆, R₇, R₁₁, R₁₂, R₁₅, R₁₆, and R₁₈ are independently selected from the group consisting of hydrogen, hydroxyl, substituted or unsubstituted (C₁-C₂₂)alkyl, substituted or unsubstituted (C₁-C₂₂)hydroxyalkyl, substituted or unsubstituted (C₁-C₂₂)alkyloxy-(C₁-C₂₂)alkyl, substituted or unsubstituted (C₁-C₂₂)alkylcarboxy-(C₁-C₂₂)alkyl, substituted or unsubstituted (C₅-C₂₅)terpenyl-carboxy-(C₁-C₂₂)alkyl, substituted or unsubstituted (C₅-C₂₅)terpenylcarbonyloxy-(C₁-C₂₂)alkyl, substituted or unsubstituted (C₅-C₂₅)terpenylcarboxamido-(C₁-C₂₂)alkyl, substituted or unsubstituted (C₅-C₂₅)terpenylamino-(C₁-C₂₂)alkyl, (C₅-C₂₅)terpenyloxyo-(C₁-C₂₂)alkyl, substituted or unsubstituted (C₁-C₂₂)alkylamino-(C₁-C₂₂)alkyl, substituted or unsubstituted (C₁-C₂₂)alkylamino-(C₁-C₂₂)alkylamino, substituted or unsubstituted (C₁-C₂₂)alkylamino-(C₁-C₂₂)alkylamino-(C₁-C₂₂)alkylamino, substituted or unsubsti-tuted (C₁-C₂₂)aminoalkyl, substituted or unsubstituted aryl, substituted or unsubstituted arylamino-(C₁-C₂₂)alkyl, substituted or unsubstituted (C₁-C₂₂)haloalkyl, substituted or unsubstituted (C₂-C₆)alkenyl, substituted or unsubstituted (C₂-C₆)alkynyl, oxo, linking group attached to a second steroid, substituted or unsubstituted (C₁-C₂₂)aminoalkylurethanyl, substituted or unsubstituted (C₂-C₂₂)aminoalkenylurethanyl, substituted or unsubstituted (C₂-C₂₂)aminoalkynylurethanyl, and substituted or unsubstituted aminoarylurethanyl, substituted or unsubstituted (C₁-C₂₂)aminoalkyloxy, substituted or unsubstituted (C₁-C₂₂)aminoalkylcarboxy, substituted or unsubstituted (C₁-C₂₂)aminoalkyloxy-(C₁-C₂₂)alkyl, substituted or unsubstituted (C₁-C₂₂)aminoalkyl-aminocarbonyl, substituted or unsubstituted (C₁-C₂₂)aminoalkylcarboxamido, substituted or unsubstituted di(C₁-C₂₂)alkylamino-(C₁-C₂₂)alkyl, H₂N—HC(Q₅)—(C═O)—O—, H₂N—HC(Q₅)—(C═O)—NH—, substituted or unsubstituted (C₁-C₂₂)azidoalkyloxy, substituted or unsubstituted (C₁-C₂₂)cyanoalkyloxy, P.G.-HN—HC(Q₅) (C═O)—O—, substituted or unsubstituted (C₁-C₂₂)guanidinoalkyloxy, substituted or unsubstituted quaternary ammonium (C₁-C₂₂)alkylcarboxy, and substituted or unsubstituted (C₁-C₂₂)guanidinoalkyl carboxy, where Q₅ is a side chain of an amino acid (including a side chain of glycine, i.e., H), and P.G. is an amino protecting group; and
R₅, R₈, R₉, R₁₀, R₁₃, R₁₄ and R₁₇ are independently deleted when one of rings A, B, C, or D is unsaturated so as to complete the valency of the carbon atom at that site, or R₅, R₈, R₉, R₁₀, R₁₃, and R₁₄ are independently selected from the group consisting of hydrogen, hydroxyl, (C₁-C₂₂)alkyl, (C₁-C₂₂)hydroxyalkyl, (C₁-C₂₂)alkyloxy-(C₁-C₂₂)alkyl, (C₁-C₂₂) aminoalkyl, aryl, (C₁-C₂₂)haloalkyl, (C₂-C₆)alkenyl, (C₂-C₆)alkynyl, oxo, a linking group attached to a second steroid, (C₁-C₂₂)aminoalkyloxy, (C₁-C₂₂)aminoalkylcarboxy, (C₁-C₂₂)aminoalkylaminocarbonyl, di(C₁-C₂₂ alkyl)amino-(C₁-C₂₂)alkyl, H₂N—HC(Q₅)—C(O)—O—, H₂N—HC(Q₅)—C(O)—N(H)—, (C₁-C₂₂)azidoalkyloxy, (C₁-C₂₂)cyanoalkyloxy, P.G.-HN—HC(Q₅)—C(O)—O—, (C₁-C₂₂)guanidinoalkyloxy, and (C₁-C₂₂)guanidinoalkylcarboxy, where Q₅ is a side chain of an amino acid, and P.G. is an amino protecting group;

provided that at least two or three of R₁₋₄, R₆, R₇, R₁₁, R₁₂, R₁₅, R₁₆, R₁₇, and R₁₈ are independently selected from the group consisting of (C₁-C₂₂)aminoalkyl, (C₁-C₂₂)aminoalkyloxy, (C₁-C₂₂)alkylcarboxy-(C₁-C₂₂)alkyl, (C₁-C₂₂)alkylamino-(C₁-C₂₂)alkylamino, (C₁-C₂₂)alkylamino-(C₁-C₂₂)alkylamino-(C₁-C₂₂)alkylamino, (C₁-C₂₂)aminoalkylcarboxy, arylamino-(C₁-C₂₂)alkyl, (C₁-C₂₂)aminoalkyloxy (C₁-C₂₂)aminoalkylaminocarbonyl, (C₁-C₂₂)aminoalkylaminocarbonyl, (C₁-C₂₂)aminoalkyl-carboxyamido, quaternary ammonium (C₁-C₂₂)alkylcarboxy, di(C₁-C₂₂ alkyl)amino-(C₁-C₂₂)alkyl, (C₁-C₂₂)aminoalkylurethanyl, (C₂-C₂₂)aminoalkenylurethanyl, (C₂-C₂₂)amino-alkynylurethanyl, aminoarylurethanyl, H₂N—HC(Q₅)—C(O)—O—, H₂N—HC(Q₅)—C(O)—N(H)—, (C₁-C₂₂)azidoalkyloxy, (C₁-C₂₂)cyanoalkyloxy, P.G.-HN—HC(Q₅)—C(O)—O—, (C₁-C₂₂) guanidinoalkyloxy, and (C₁-C₂₂)guanidinoalkylcarboxy.

In embodiments, at least one of R₁-R₁₈, preferably R₁₈, can have the following bioresorbable mono- or diglyceride structure:

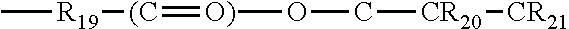

where R₁₉ is omitted or is selected from alkyl, alkenyl, alkynyl, and aryl, and R₂₀ and R₂₁ are independently selected from hydroxy and (C₂-C₂₂)alkylcarboxy, provided that at least one of R₂₀ or R₂₁ is (C₂-C₂₂)alkylcarboxy, the (C₂-C₂₂)alkylcarboxy preferably having an even number of carbons. The glyceride portion of foregoing structure forms bioresorbable glycerin and fatty acid(s) as degradation product (e.g., by hydrolysis of ester groups in the glyceride structure).

In some embodiments, R₁₈ can have the following bioresorbable mixed diglyceride structure:

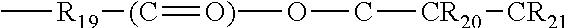

where R₁₉ is omitted or is selected from alkyl, alkenyl, alkynyl, and aryl, R₂₀ is a (C₂-C₂₂)alkylcarboxy, the (C₂-C₂₂)alkylcarboxy preferably having an even number of carbons, and R₂₁ can have the following aminoalkylcarboxy structure:

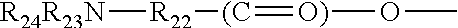

where R₂₂ is a substituted or unsubstituted alkyl and R₂₃ and R₂₄ are independently selected from hydrogen, alkyl, alkenyl, alkynyl, and aryl. R₂₂ is preferably an ester group of an amino acid, such as beta-alanine, which forms a bioresorbable amino acid (e.g., beta-alanine) as degradation product (e.g., by hydrolysis of the ester groups at the C₂₄). The glyceride portion forms bioresorbable glycerin, a fatty acid, and an amino acid as degradation products (e.g., by hydrolysis of ester groups in the glyceride structure).

In some embodiments, at least one of R₁-R₁₈, preferably at least one of R₃, R₇ and R₁₂, can have the following bioresorbable aminoalkylcarboxy or aminoalkylcarboxamido structure:

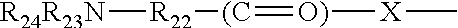

where R₂₂ is a substituted or unsubstituted alkyl, X is oxygen or nitrogen, and R₂₃ and R₂₄ are independently selected from hydrogen, alkyl, alkenyl, alkynyl, and aryl. At least one of R₃, R₇ and R₁₂, preferably two or three of R₃, R₇ and R₁₂, is/are an ester group of one or more amino acids, such as beta-alanine, which forms a bioresorbable amino acid (e.g., beta-alanine) as degradation product (e.g., by hydrolysis of the ester group(s) at the C₃, C₇ and/or C₁₂ position(s)). Alternatively, the aminoalkyl portion of at least one of R₃, R₇ and R₁₂ can be attached to one or more of the C₃, C₇ and/or C₁₂ positions of the sterol backbone (or elsewhere) by other linkages, such as amide or ether linkage.

In some embodiments, bioresorbable mono- and diglyceride CSA compounds can have a chiral center, such as in the glyceryl moiety, so as to form enantiomers that can be isolated rather than forming a racemic mixture. Unless otherwise specified, the examples of CSA compounds disclosed herein can be non-chiral, R- and S-enantiomers forming a racemic mixture, the R-enantiomer, or the S-enantiomer.

Non-limiting examples of bioresorbable monoglyceride CSA compounds that form bioresorbable degradation products by hydrolysis of ester groups are CSA-4108, CSA-4110 (racemic mixture), CSA-4110R (R-enantiomer), CSA-4110S (S-enantiomer), CSA-4112, CSA-4114, and salts thereof (see FIG. 1D). Non-limiting examples of bioresorbable diglyceride CSA compounds that form bioresorbable degradation products by hydrolysis of ester groups are CSA-4204, CSA-4206, CSA-4208, CSA-4210, and CSA-4310, and salts thereof (see FIG. 1D).

In embodiments, R₁, R₂, R₄, R₅, R₆, R₈, R₉, R₁₀, R₁₁, R₁₃, R₁₄, R₁₅, R₁₆, and R₁₇ are independently selected from the group consisting of hydrogen and unsubstituted (C₁-C₆)alkyl.

In embodiments, R₁, R₂, R₄, R₅, R₆, R₈, R₁₀, R₁₁, R₁₄, R₁₆, and R₁₇ are each hydrogen and R₉ and R₁₃ are each methyl.

In embodiments, R₃, R₇, R₁₂, and R₁₈ are independently selected from the group consisting of hydrogen, (C₁-C₆)alkyl, (C₁-C₆)hydroxyalkyl, (C₁-C₁₆)alkyloxy-(C₁-C₅)alkyl, (C₁-C₁₆)alkylcarboxy-(C₁-C₅)alkyl, (C₁-C₁₆)alkylamino-(C₁-C₅)alkyl, (C₁-C₁₆)alkylamino-(C₁-C₅)alkylamino, (C₁-C₁₆)alkylamino-(C₁-C₁₆)alkylamino-(C₁-C₅)alkylamino, (C₅-C₂₅)terpenylcarboxy-(C₁-C₅)alkyl, (C₅-C₂₅)terpenylcarbonyloxy-(C₁-C₅)alkyl, (C₅-C₂₅)terpenylcarboxamido-(C₁-C₅)alkyl, (C₅-C₂₅)terpenylamino-(C₁-C₅)alkyl, (C₅-C₂₅)terpenyloxyo-(C₁-C₅)alkyl, (C₁-C₆)aminoalkylurethanyl, (C₂-C₆)aminoalkenylurethanyl, (C₂-C₆)aminoalkynylurethanyl, aminoarylurethanyl, (C₁-C₁₆)aminoalkyl, arylamino-(C₁-C₅)alkyl, (C₁-C₅)aminoalkyloxy, (C₁-C₁₆)aminoalkyl-oxy-(C₁-C₅)alkyl, (C₁-C₅)aminoalkylcarboxy, (C₁-C₅)aminoalkyl-aminocarbonyl, (C₁-C₅)aminoalkylcarbox-amido, di(C₁-C₅ alkyl)amino-(C₁-C₅)alkyl, (C₁-C₅)guanidino-alkyloxy, quaternary ammonium (C₁-C₁₆)alkylcarboxy, and unsubstituted (C₁-C₁₆)guanidinoalkylcarboxy.

In embodiments, R₁, R₂, R₄, R₅, R₆, R₈, R₁₀, R₁₁, R₁₄, R₁₆, and R₁₇ are each hydrogen; and R₉ and R₁₃ are each methyl.

In embodiments, R₃, R₇, R₁₂, and R₁₈ are independently selected from the group consisting of aminoalkyloxy, aminoalkylcarboxy, alkylaminoalkyl, alkoxycarbonylalkyl, alkylcarbonylalkyl, di(alkyl)aminoalkyl, alkylcarboxyalkyl, hydroxyalkyl, terpenylcarboxyalkyl, terpenylcarbonyloxyalkyl, terpenylcarboxamido-alkyl, terpenylamino-alkyl, terpenyloxyoalkyl, aminoalkylurethanyl, aminoalkenylurethanyl, aminoalkynyl-urethanyl, and aminoarylurethanyl.

In embodiments, R₃, R₇, and R₁₂ are independently selected from the group consisting of aminoalkyloxy, aminoalkylcarboxy, aminoalkylurethanyl, aminoalkenyl-urethanyl, aminoalkynylurethanyl, and aminoarylurethanyl.

In embodiments, R₁₈ is selected from the group consisting of alkylaminoalkyl, alkoxycarbonylalkyl, alkylcarbonyloxyalkyl, alkylcarbonylalkyl, di(alkyl)aminoalkyl, alkylcarboxyalkyl, hydroxyalkyl, terpenylcarboxyalkyl, terpenylcarbonyloxyalkyl, terpenylcarboxamido-alkyl, terpenylaminoalkyl, and terpenyloxyoalkyl.

In embodiments, one or more of rings A, B, C, and D is heterocyclic.

In embodiments, rings A, B, C, and D are non-heterocyclic.

The compounds and compositions disclosed herein are optionally prepared as salts, which advantageously makes them cationic when one or more amine groups is/are protonated. “Salt” as used herein is a broad term, and is to be given its ordinary and customary meaning to a skilled artisan (and is not to be limited to a special or customized meaning), and refers without limitation to a salt of a compound. In embodiments, the salt is an acid addition salt of the compound. Salts can be obtained by reacting a compound with inorganic acids such as hydrohalic acid (e.g., hydrochloric acid or hydrobromic acid), sulfuric acid, nitric acid, phosphoric acid, and phosphonic acid. Salts can also be obtained by reacting a compound with an organic acid such as aliphatic or aromatic carboxylic or sulfonic acids, sulfinic acids, for example formic acid, acetic acid, propionic acid, glycolic acid, pyruvic acid, malonic acid, maleic acid, fumaric acid, trifluoroacetic acid, benzoic acid, cinnamic acid, mandelic acid, succinic acid, lactic acid, malic acid, tartaric acid, citric acid, ascorbic acid, nicotinic acid, methanesulfonic acid, ethanesulfonic acid, p-toluensulfonic acid, salicylic acid, stearic acid, muconic acid, butyric acid, phenylacetic acid, phenylbutyric acid, valproic acid, 1,2-ethanedisulfonic acid, 2-hydroxyethanesulfonic acid, benzenesulfonic acid, 2-naphthalenesulfonic acid, or 1,5-naphthalenedisulfonic acid (NDSA). Salts can also be obtained by reacting a compound with a base to form a salt such as an ammonium salt, an alkali metal salt, such as a lithium, sodium or a potassium salt, an alkaline earth metal salt, such as a calcium, magnesium or aluminum salt, a salt of organic bases such as dicyclohexylamine, N-methyl-D-glucamine, tris(hydroxymethyl)methylamine, C₁-C₇ alkylamine, cyclohexyl-amine, dicyclohexylamine, tricthanolamine, ethylenediamine, ethanolamine, diethanolamine, triethanolamine, tromethamine, and salts with amino acids such as arginine and lysine; or a salt of an inorganic base, such as aluminum hydroxide, calcium hydroxide, potassium hydroxide, sodium carbonate, sodium hydroxide, or the like.

In embodiments, the salt is a hydrochloride salt. In embodiments, the salt is a mono-hydrochloride salt, a di-hydrochloride salt, a tri-hydrochloride salt, or a tetra-hydrochloride salt. Additional examples of salts include sulfuric acid addition salts, sulfonic acid addition salts, disulfonic acid addition salts, 1,5-naphthalenedisulfonic acid addition salts, sulfate salts, and bisulfate salts.

“R” groups such as, without limitation, R₁, R₂, R₃, R₄, R₅, R₆. R₇, R₈, R₉, R₁₀, R₁₁, R₁₂, R₁₃, R₁₄, R₁₅. R₁₆, R₁₇, and R₁₈, represent substituents that can be attached to the sterol backbone. Unless otherwise specified, an R group may be substituted or unsubstituted.

A “ring” can be heterocyclic or carbocyclic. “Saturated” means a ring in which each atom is either hydrogenated or substituted such that the valency of each atom is filled. “Unsaturated” means a ring where the valency of each atom of the ring may not be filled with hydrogen or other substituents. For example, adjacent carbon atoms in a fused ring can be double bound to each other. Unsaturation can also include deleting at least one of the following pairs and completing the valency of the ring carbon atoms at these deleted positions with a double bond, such as R₈ and R₉; R₈ and R₁₀; and R₁₃ and R₁₄.

Where a group is “substituted” it may be substituted with one, two, three or more of the indicated substituents, which may be the same or different, each replacing a hydrogen atom. If no substituents are indicated, the indicated “substituted” group may be substituted with one or more groups individually and independently selected from alkyl, alkenyl, alkynyl, cycloalkyl, cycloalkenyl, cycloalkynyl, acylalkyl, alkoxyalkyl, aminoalkyl, amino acid, aryl, heteroaryl, heteroalicyclyl, aralkyl, heteroaralkyl, (heteroalicyclyl)alkyl, hydroxy, protected hydroxyl, alkoxy, aryloxy, acyl, mercapto, alkylthio, arylthio, cyano, halogen (e.g., F, Cl, Br, and I), thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, oxo, silyl, sulfenyl, sulfinyl, sulfonyl, haloalkyl, haloalkoxy, trihalomethanesulfonyl, trihalomethanesulfonamido, an amino, a mono-substituted amino group and a di-substituted amino group, RₐO(CH₂)ₘO—, R_{b}(CH₂)ₙO—, R_{c}C(O)O(CH₂)ₚO—, and protected derivatives thereof. The substituent may be attached to the group at more than one attachment point. For example, an aryl group may be substituted with a heteroaryl group at two attachment points to form a fused multicyclic aromatic ring system. Biphenyl and naphthalene are two examples of an aryl group that is substituted with a second aryl group. A group that is not specifically labeled as substituted or unsubstituted may be considered to be either substituted or unsubstituted.

The terms “Ca” or “Ca to Ch” in which “a” and “b” are integers refer to the number of carbon atoms in an alkyl, alkenyl or alkynyl group, or the number of carbon atoms in the ring of a cycloalkyl, cycloalkenyl, cycloalkynyl, aryl, heteroaryl or heteroalicyclyl group. That is, the alkyl, alkenyl, alkynyl, ring of the cycloalkyl, ring of the cycloalkenyl, ring of the cycloalkynyl, ring of the aryl, ring of the heteroaryl or ring of the heteroalicyclyl can contain from “a” to “b”, inclusive, carbon atoms. Thus, for example, a “C₁ to C₄ alkyl” group refers to all alkyl groups having 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)—, (CH₃)₂CHCH₂— and (CH₃)₃C—. If no “a” and “b” are designated with regard to an alkyl, alkenyl, alkynyl, cycloalkyl cycloalkenyl, cycloalkynyl, aryl, heteroaryl or heteroalicyclyl group, the broadest range described in these definitions is to be assumed.

“Alkyl” means a straight or branched hydrocarbon chain that comprises a fully saturated (no double or triple bonds) hydrocarbon group. The alkyl group may have 1 to 25 carbon atoms (whenever it appears herein, a numerical range such as “1 to 25” refers to each integer in the given range; e.g., “1 to 25 carbon atoms” means that the alkyl group may consist of 1 carbon atom, 2 carbon atoms, 3 carbon atoms, etc., up to and including 25 carbon atoms, although the present definition also covers the occurrence of the term “alkyl” where no numerical range is designated). The alkyl group may also be a medium size alkyl having 1 to 15 carbon atoms. The alkyl group could also be a lower alkyl having 1 to 6 carbon atoms. The alkyl group of the compounds may be designated as “C₄” or “C₁-C₄ alkyl” or similar designations. By way of example only, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl and hexyl. The alkyl group may be substituted or unsubstituted.

“Alkenyl” means an alkyl group that contains in the straight or branched hydrocarbon chain one or more double bonds. The alkenyl group may have 2 to 25 carbon atoms (whenever it appears herein, a numerical range such as “2 to 25” refers to each integer in the given range; e.g., “2 to 25 carbon atoms” means that the alkenyl group may consist of 2, 3, or 4 carbon atoms, etc., up to and including 25 carbon atoms, although the present definition also covers the occurrence of the term “alkenyl” where no numerical range is designated). The alkenyl group may also be a medium size alkenyl having 2 to 15 carbon atoms. The alkenyl group could also be a lower alkenyl having 1 to 6 carbon atoms. The alkenyl group of the compounds may be designated as “C₄” or “C₂-C₄ alkenyl” or similar designations. An alkenyl group may be unsubstituted or substituted.

“Alkynyl” means an alkyl group that contains in the straight or branched hydrocarbon chain one or more triple bonds. The alkynyl group may have 2 to 25 carbon atoms (whenever it appears herein, a numerical range such as “2 to 25” refers to each integer in the given range; e.g., “2 to 25 carbon atoms” means that the alkynyl group may consist of 2, 3, or 4 carbon atoms, etc., up to and including 25 carbon atoms, although the present definition also covers the occurrence of the term “alkynyl” where no numerical range is designated). The alkynyl group may also be a medium size alkynyl having 2 to 15 carbon atoms. The alkynyl group could also be a lower alkynyl having 2 to 6 carbon atoms. The alkynyl group of the compounds may be designated as “C₄” or “C₂-C₄ alkynyl” or similar designations. An alkynyl group may be unsubstituted or substituted.

“Aryl” means a carbocyclic (all carbon) monocyclic or multicyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group, or a C₆ aryl group (although the definition of C₆-C₁₀ aryl covers the occurrence of “aryl” when no numerical range is designated). Examples of aryl groups include, but are not limited to, benzene, naphthalene and azulene. An aryl group may be substituted or unsubstituted.

“Aralkyl” and “aryl (alkyl)” mean an aryl group connected, as a substituent, via a lower alkylene group. The aralkyl group may have 6 to 20 carbon atoms (whenever it appears herein, a numerical range such as “6 to 20” refers to each integer in the given range; e.g., “6 to 20 carbon atoms” means that the aralkyl group may consist of 6 carbon atom, 7 carbon atoms, 8 carbon atoms, etc., up to and including 20 carbon atoms, although the present definition also covers the occurrence of the term “aralkyl” where no numerical range is designated). The lower alkylene and aryl group of an aralkyl may be substituted or unsubstituted. Examples include but are not limited to benzyl, 2-phenylalkyl, 3-phenylalkyl, and naphthylalkyl.

“Lower alkylene groups” mean a C₁-C₂₅ straight-chained alkyl tethering groups, such as —CH₂— tethering groups, forming bonds to connect molecular fragments via their terminal carbon atoms. Examples include but are not limited to methylene (—CH₂—), ethylene (—CH₂CH₂—), propylene (—CH₂CH₂CH₂—), and butylene (—CH₂CH₂CH₂CH₂—). A lower alkylene group can be substituted by replacing one or more hydrogen of the lower alkylene group with a substituent(s) listed under the definition of “substituted.”

“Cycloalkyl” means a completely saturated (no double or triple bonds) mono- or multi-cyclic hydrocarbon ring system. When composed of two or more rings, the rings may be joined together in a fused fashion. Cycloalkyl groups can contain 3 to 10 atoms in the ring(s) or 3 to 8 atoms in the ring(s). A cycloalkyl group may be unsubstituted or substituted. Typical cycloalkyl groups include, but are in no way limited to, cyclopropyl, cyclobutyl, cyclopentyl, cyclohexyl, cycloheptyl and cyclooctyl.

“Cycloalkenyl” means a mono- or multi-cyclic hydrocarbon ring system that contains one or more double bonds in at least one ring; although, if there is more than one, the double bonds cannot form a fully delocalized pi-electron system throughout all the rings (otherwise the group would be “aryl,” as defined herein). When composed of two or more rings, the rings may be connected together in a fused fashion. A cycloalkenyl group may be unsubstituted or substituted.

“Cycloalkynyl” means a mono- or multi-cyclic hydrocarbon ring system that contains one or more triple bonds in at least one ring. If there is more than one triple bond, the triple bonds cannot form a fully delocalized pi-electron system throughout all the rings. When composed of two or more rings, the rings may be joined together in a fused fashion. A cycloalkynyl group may be unsubstituted or substituted.

“Alkoxy” or “alkyloxy” mean the formula-OR wherein R is an alkyl, an alkenyl, an alkynyl, a cycloalkyl, a cycloalkenyl or a cycloalkynyl as defined above. Examples of alkoxys are methoxy, ethoxy, n-propoxy, 1-methylethoxy (isopropoxy), n-butoxy, iso-butoxy, sec-butoxy and tert-butoxy. An alkoxy may be substituted or unsubstituted.

“Acyl” means a hydrogen, alkyl, alkenyl, alkynyl, aryl, or heteroaryl connected, as substituents, via a carbonyl group, such as —(C═O)—R. Examples include formyl, acetyl, propanoyl, benzoyl, and acryl. An acyl may be substituted or unsubstituted.

“Alkoxyalkyl” or “alkyloxyalkyl” mean an alkoxy group connected, as a substituent, via a lower alkylene group. Examples include alkyl-O-alkyl- and alkoxy-alkyl with the terms alkyl and alkoxy defined herein.

“Hydroxyalkyl” means an alkyl group in which one or more of the hydrogen atoms are replaced by a hydroxy group. Exemplary hydroxyalkyl groups include but are not limited to, 2-hydroxyethyl, 3-hydroxypropyl, 2-hydroxypropyl, and 2,2-dihydroxyethyl. A hydroxyalkyl may be substituted or unsubstituted.

“Haloalkyl” means an alkyl group in which one or more of the hydrogen atoms are replaced by a halogen (e.g., mono-haloalkyl, di-haloalkyl and tri-haloalkyl). Examples include chloromethyl, fluoromethyl, difluoromethyl, trifluoromethyl and 1-chloro-2-fluoromethyl, 2-fluoroisobutyl. A haloalkyl may be substituted or unsubstituted.

“Amino” means “—NH₂”.

“Hydroxy” means “—OH”.

“Cyano” means “—CN”.

“Carbonyl” or “oxo” mean “—C═O”.

“Azido” means “—N₃”.

“Aminoalkyl” means an amino group connected, as a substituent, via a lower alkylene group. Examples include H₂N-alkyl- with the term alkyl defined herein.

“Alkylcarboxyalkyl” means an alkyl group connected, as a substituent, to a carboxy group that is connected, as a substituent, to an alkyl group. Examples include alkyl-(C═O)—O-alkyl- and alkyl-O—(C═O)-alkyl- with the term alkyl as defined herein.

“Alkylaminoalkyl” means an alkyl group connected, as a substituent, to an amino group that is connected, as a substituent, to an alkyl group. Examples include alkyl-NH-alkyl with the term alkyl as defined herein.

“Dialkylaminoalkyl” and “di(alkyl)aminoalkyl” mean two alkyl groups connected, each as a substituent, to an amino group that is connected, as a substituent, to an alkyl group. Examples include

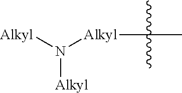

with the term alkyl as defined herein.

“Alkylaminoalkylamino” means an alkyl group connected, as a substituent, to an amino group that is connected, as a substituent, to an alkyl group that is connected, as a substituent, to an amino group. Examples include alkyl-NH-alkyl-NH— with the term alkyl as defined herein.

“Alkylaminoalkylaminoalkylamino” means an alkyl group connected, as a substituent, to an amino group that is connected, as a substituent, to an alkyl group that is connected, as a substituent, to an amino group that is connected, as a substituent, to an alkyl group. Examples include alkyl-NH-alkyl-NH-alkyl- with the term alkyl as defined herein.

“Arylaminoalkyl” means an aryl group connected, as a substituent, to an amino group that is connected, as a substituent, to an alkyl group. Examples include aryl-NH-alkyl with the terms aryl and alkyl as defined herein.

“Aminoalkyloxy” means an amino group connected, as a substituent, to an alkyloxy group. Examples include H₂N-alkyl-O— and H₂N-alkoxy- with the terms alkyl and alkoxy as defined herein.

“Aminoalkyloxyalkyl” means an amino group connected, as a substituent, to an alkyloxy group connected, as a substituent, to an alkyl group. Examples include H₂N-alkyl-O-alkyl- and H₂N-alkoxy-alkyl- with the terms alkyl and alkoxy as defined herein.

“Aminoalkylcarboxy” means an amino group connected, as a substituent, to an alkyl group connected, as a substituent, to a carboxy group. Examples include H₂N-alkyl-(C═O)—O— and H₂N-alkyl-O—(C═O)— with the term alkyl as defined herein.

“Aminoalkylaminocarbonyl” means an amino group connected, as a substituent, to an alkyl group connected, as a substituent, to an amino group connected, as a substituent, to a carbonyl group. Examples include H₂N-alkyl-NH—(C═O)— with the term alkyl as defined herein.

“Aminoalkylcarboxamido” means an amino group connected, as a substituent, to an alkyl group connected, as a substituent, to a carbonyl group connected, as a substituent to an amino group. Examples include H₂N-alkyl-(C═O)—NH— and H₂N-alkyl-NH—(C—O) with the term alkyl as defined herein.

“Azidoalkyloxy” means an azido group connected as a substituent, to an alkyloxy group. Examples include N₃-alkyl-O— and N₃-alkoxy- with the terms alkyl and alkoxy as defined herein.

“Cyanoalkyloxy” means a cyano group connected as a substituent, to an alkyloxy group. Examples include NC-alkyl-O— and NC-alkoxy- with the terms alkyl and alkoxy as defined herein.

“Sulfenyl” means “—SR” in which R can be hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. A sulfenyl may be substituted or unsubstituted.

“Sulfinyl” means “—(S═O)—R” in which R can be the same as defined with respect to sulfenyl. A sulfinyl may be substituted or unsubstituted.

“Sulfonyl” means “—(S═O)—OR” in which R can be the same as defined with respect to sulfenyl. A sulfonyl may be substituted or unsubstituted.

“O-carboxy” means “R—(C═O)—O—” in which R can be hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl, as defined herein. An O-carboxy may be substituted or unsubstituted.

“Ester” and “C-carboxy” mean “—(C═O)—OR” in which R can be the same as defined with respect to O-carboxy. An ester and C-carboxy may be substituted or unsubstituted.

“Thiocarbonyl” means “—(C═S)—R” in which R can be the same as defined with respect to O-carboxy. A thiocarbonyl may be substituted or unsubstituted.

“Trihalomethanesulfonyl” means “X₃CSO₂—” wherein X is a halogen.

“S-sulfonamido” means “—SO₂N (RARB)” in which RA and RB can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. An S-sulfonamido may be substituted or unsubstituted.

“N-sulfonamido” means “RSO₂N(RA)-” in which R and RA can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. An N-sulfonamido may be substituted or unsubstituted.

“O-carbamyl” and “urethanyl” mean “—O—(C—O)—N(RARB)” in which RA and RB can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. An O-carbamyl or urethanyl may be substituted or unsubstituted.

“N-carbamyl” means “RO—(C—O)—N(RA)-” in which R and RA can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. An N-carbamyl may be substituted or unsubstituted.

“O-thiocarbamyl” means “—O—(C═S)—N(RARB)” in which RA and RB can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. An O-thiocarbamyl may be substituted or unsubstituted.

“N-thiocarbamyl” means “RO—(C═S)—N(RA)-” in which R and RA can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. An N-thiocarbamyl may be substituted or unsubstituted.

“C-amido” means “—(C—O)—N(RARB)” in which RA and RB are independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. A C-amido may be substituted or unsubstituted.

“N-amido” means “R—(C═O)—N(RA)-” in which R and RA are independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. An N-amido may be substituted or unsubstituted.

“Guanidinoalkyloxy” means a guanidinyl group connected, as a substituent, to an alkyloxy group. Examples are

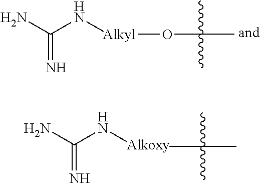

with the terms alkyl and alkoxy as defined herein.

“Guanidinoalkylcarboxy” means a guanidinyl group connected, as a substituent, to an alkyl group connected, as a substituent, to a carboxy group. Examples are

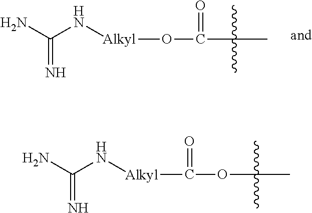

with the term alkyl as defined herein.

“Quaternary ammonium alkylcarboxy” means a quaternized amino group connected, as a substituent, to an alkyl group connected, as a substituent, to a carboxy group. Examples are

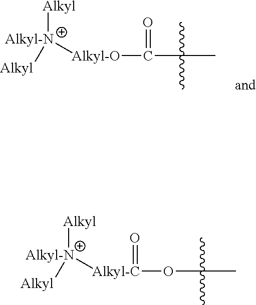

with the term alkyl as defined herein.

“Halogen atom” and “halogen” mean any one of the radio-stable atoms of column 7 of the Periodic Table of the Elements, such as, fluorine, chlorine, bromine and iodine.

Where the number of substituents is not specified (e.g. haloalkyl), there may be one or more substituents present. For example, “haloalkyl” may include one or more of the same or different halogens.

“Amino acid” means any amino acid (both standard and non-standard amino acids), including, but not limited to, α-amino acids, β-amino acids, γ-amino acids and δ-amino acids. Examples of suitable amino acids include, but are not limited to, alanine, asparagine, aspartate, cysteine, glutamate, glutamine, glycine, proline, serine, tyrosine, arginine, histidine, isoleucine, leucine, lysine, methionine, phenylalanine, threonine, tryptophan and valine. Additional examples of suitable amino acids include, but are not limited to, ornithine, hypusine, 2-aminoisobutyric acid, dehydroalanine, γ-aminobutyric acid, citrulline, β-alanine, α-ethyl-glycine, α-propyl-glycine and norleucine.

A “linking group” is a divalent moiety used to link one steroid to another steroid. In embodiments, the linking group is used to link a first CSA with a second CSA (which may be the same or different). An example of a linking group is (C₁-C₁₀)alkyloxy-(C₁-C₁₀)alkyl.

“P.G.” or “protecting group” or “protecting groups” mean any atom or group of atoms that is added to a molecule in order to prevent existing groups in the molecule from undergoing unwanted chemical reactions. Examples of protecting group moieties are described in T. W. Greene and P. G. M. Wuts, Protective Groups in Organic Synthesis, 3. Ed. John Wiley & Sons, 1999, and in J. F. W. McOmie, Protective Groups in Organic Chemistry Plenum Press, 1973, both of which are hereby incorporated by reference for the limited purpose of disclosing suitable protecting groups. The protecting group moiety may be chosen in such a way, that they are stable to certain reaction conditions and readily removed at a convenient stage using methodology known from the art. A non-limiting list of protecting groups include benzyl; substituted benzyl; alkylcarbonyls and alkoxycarbonyls (e.g., t-butoxycarbonyl (BOC), acetyl, or isobutyryl); arylalkylcarbonyls and arylalkoxycarbonyls (e.g., benzyloxycarbonyl); substituted methyl ether (e.g. methoxymethyl ether); substituted ethyl ether; substituted benzyl ether; tetrahydropyranyl ether; silyls (e.g., trimethylsilyl, triethylsilyl, triisopropylsilyl, t-butyldimethylsilyl, tri-iso-propylsilyloxymethyl, [2-(trimethylsilyl) ethoxy]methyl or t-butyldiphenylsilyl); esters (e.g. benzoate ester); carbonates (e.g. methoxymethylcarbonate); sulfonates (e.g. tosylate or mesylate); acyclic ketal (e.g. dimethyl acetal); cyclic ketals (e.g., 1,3-dioxane, 1,3-dioxolanes, and those described herein); acyclic acetal; cyclic acetal (e.g., those described herein); acyclic hemiacetal; cyclic hemiacetal; cyclic dithioketals (e.g., 1,3-dithiane or 1,3-dithiolane); orthoesters (e.g., those described herein) and triarylmethyl groups (e.g., trityl; monomethoxytrityl (MMTr); 4,4′-dimethoxytrityl (DMTr); 4,4′,4″-trimethoxytrityl (TMTr); and those described herein). Amino-protecting groups are known to those skilled in the art. In general, the species of protecting group is not critical, provided that it is stable to the conditions of any subsequent reaction(s) on other positions of the compound and can be removed at the appropriate point without adversely affecting the remainder of the molecule. In addition, a protecting group may be substituted for another after substantive synthetic transformations are complete. Clearly, where a compound differs from a compound disclosed herein only in that one or more protecting groups of the disclosed compound has been substituted with a different protecting group, that compound is within the disclosure.

Additional features and advantages will be set forth in part in the description that follows, and in part will be obvious from the description, or may be learned by practice of the embodiments disclosed herein. It is to be understood that both the foregoing brief summary and the following detailed description are exemplary and not restrictive of the embodiments disclosed herein or as claimed.

1. An implantable medical device configured to elute one or more cationic steroidal antimicrobial (CSA) compounds, comprising:
a base substrate forming at least a portion of the implantable medical device;
a multi-layered coating disposed on the base substrate, the multi-layered coating comprising
(i) a CSA coating layer disposed over the base substrate, the CSA coating layer comprising a polymer in which one or more CSA compounds are distributed, and
(ii) a top coating layer disposed over the CSA coating layer, the top coating layer being free of CSA compounds,
wherein when the implantable medical device is exposed to a physiological environment, the top coating layer provides
(i) more sustained elution of the one or more CSA compounds, as compared to the implantable medical device without the top coating layer, and/or
(ii) reduced swelling of the CSA coating layer, as compared to the implantable medical device without the top coating layer.

2. The implantable medical device of claim 1, wherein the implantable medical device comprises a catheter.

3. The implantable medical device of claim 2, wherein the catheter is a peripherally inserted central catheter (PICC).

4. The implantable medical device of claim 1, wherein the one or more CSA compounds comprise a CSA compound that includes cationic groups linked to a sterol backbone with non-hydrolysable linkages.

5. The implantable medical device of claim 4, wherein the non-hydrolysable linkages are ether linkages.

6. The implantable medical device of claim 5, wherein the one or more CSA compounds comprise CSA-131.

7. The implantable medical device of claim 1, wherein the one or more CSA compounds comprise a CSA compound in the form of a sulfonic acid addition salt.

8. The implantable medical device of claim 7, wherein the sulfonic acid addition salt is a naphthalenedisulfonic acid (NDSA) salt.

9. The implantable medical device of claim 8, wherein the NDSA salt is a 1,5-NDSA salt.

10. The implantable medical device of claim 1, wherein the CSA coating layer, the top coating layer, or both comprise a polyurethane, silicone elastomer, polyethylene glycol (PEG), polyvinyl alcohol (PVA), fluoropolymer (e.g., polytetrafluoroethylene), polyvinylpyrrolidone (PVP), polysulfone (PSU), or combination thereof.

11. The implantable medical device of claim 1, wherein the CSA coating layer is formed from a CSA coating composition that includes the one or more CSA compounds at a concentration of 10% (w/w) to 60% (w/w), based on weight of total solids of the CSA coating composition.

12. The implantable medical device of claim 1, wherein the multi-layered coating further comprises a primer layer disposed between the CSA coating layer and the base substrate.

13. The implantable medical device of claim 12, wherein the primer layer comprises silicone.

14. The implantable medical device of claim 1, wherein a ratio of thickness of the CSA coating layer to thickness of the top coating layer is 0.75 to 3.

15. A method of manufacturing an implantable medical device configured to elute one or more cationic steroidal antimicrobial (CSA) compounds, comprising:
applying a CSA coating layer over a base substrate, the base substrate forming at least a portion of the implantable medical device, the CSA coating layer comprising a polymer in which one or more CSA compounds are distributed; and
applying a top coating layer over the CSA coating layer, the top coating layer being free of CSA compounds,
wherein when the implantable medical device is exposed to a physiological environment, the top coating layer provides
(i) more sustained elution of the one or more CSA compounds, as compared to the implantable medical device without the top coating layer, and/or
(ii) reduced swelling of the CSA coating layer, as compared to the implantable medical device without the top coating layer.

16. The method of claim 15, wherein the CSA coating layer, the top coating layer, or both are applied via dip coating, spray coating, spin coating, electrospinning, electrophoretic deposition (EPD), solvent casting, thermal spraying, or combination thereof.

17. The method of claim 15, wherein the multi-layer coating provides multiple weeks of at least a 50% reduction in planktonic and/or biofilm microbial growth on the implantable medical device, when exposed to a physiological environment, as compared to the implantable medical device without a CSA coating layer.

18. The method of claim 15, wherein the implantable medical device comprises a peripherally inserted central catheter (PICC).

19. The method of claim 15, wherein the one or more CSA compounds comprise a CSA compound that includes cationic groups linked to a sterol backbone with non-hydrolysable linkages.

20. The method of claim 15, wherein the one or more CSA compounds comprise a CSA compound in the form of a naphthalenedisulfonic acid (NDSA) salt.
